(19) United States
(12) Patent Application Publication
(10) Pub. No.: US 2026/0294563 A1
(43) Pub. Date: Oct. 1, 2026

(54) SYSTEMS AND METHODS FOR IMPLANTING NEURAL ELECTRODES

(71) Applicant: William Marsh Rice University, Houston, TX (US)

(72) Inventors: Lan Luan, Houston, TX (US); Chong Xie, Houston, TX (US); Yu Wu, Houston, TX (US); Romain Albouy, Houston, TX (US); Weinan Wang, Houston, TX (US)

(73) Assignee: William Marsh Rice University, Houston, TX (US)

(21) Appl. No.: 19/489,112

(22) PCT Filed: May 31, 2024
(86) PCT No.: PCT/US2024/032106

§ 371 Date: Dec. 1, 2025

Related U.S. Application Data
(60) Provisional application No. 63/505,548, filed on Jun. 1, 2023.

(51) Int. Cl. A61B 34/32; A61B 17/00; A61B 17/34; A61B 34/00; A61B 34/10; A61B 90/00; A61B 90/20; A61B 90/30

(52) U.S. Cl. CPC A61B 34/32; A61B 17/3403; A61B 34/10; A61B 34/25; A61B 90/20; A61B 90/30; A61B 90/37; A61B 2017/00057; A61B 2017/00526; A61B 2017/3409; A61B 2034/107; A61B 2090/373

(57) ABSTRACT

A system for implanting flexible neural electrodes includes an insertion robot and a controller. The insertion robot includes a base and a plurality of manipulator arms each configured to support a micro needle. The insertion robot further includes a plurality of actuators supported by the base where each manipulator arm is translatable in three orthogonal directions by a corresponding actuator of the plurality of actuators. The plurality of manipulator arms is configured to reduce an area spanned by its distal ends relative to an area, spanned by the plurality of actuators. The controller includes one or more processors and is in electrical communication with the insertion robot. The controller is configured to control each manipulator arm in the plurality of manipulator arms to implant, with the micro needle, a flexible neural electrode into neural tissue, with at least some insertions being performed in parallel.

### STATEMENT REGARDING FEDERALLY SPONSORED RESEARCH

This invention was made with government support under Grant No. U01NS115588 and Grant No. R01NS102917 awarded by the National Institute of Health. The government has certain rights in the invention.

### BACKGROUND

Electrodes may be used to establish an electrical interface with neural tissue. Such electrodes have various applications, and may be used for brain-computer interfaces, diagnosis and/or treatment of neurological disorders, neuroscience research, etc. Frequently, it may be desirable to simultaneously use multiple or even many electrodes, e.g., 10s, 100s, 1000s of electrodes, when recording from neural tissue and/or stimulating the neural tissue. The implantation of multiple electrodes can be tedious, time-consuming, and risky.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a system for implanting flexible neural electrodes (e.g., nanoelectronic threads (NETs)) including an insertion robot and a controller. The insertion robot includes a base and a plurality of manipulator arms each configured to support a micro needle. The insertion robot further includes a plurality of actuators supported by the base where each manipulator arm is translatable in three orthogonal directions by a corresponding actuator of the plurality of actuators. The plurality of manipulator arms is configured to reduce an area spanned by its distal ends relative to an area spanned by the plurality of actuators. The controller includes one or more processors and is in electrical communication with the insertion robot. The controller is configured to control each manipulator arm in the plurality of manipulator arms to implant, with the micro needle, a flexible neural electrode into neural tissue, with at least some insertions being performed in parallel.

In one aspect, embodiments disclosed herein relate to a method for implanting flexible neural electrodes (e.g., nanoelectronic threads (NETs)). The method includes scanning, with a laser scanner, a craniotomy site with exposed neural tissue and proximate micro needles, forming a laser scan and determining, based on the laser scan, coordinates of the micro needles with respect to a surface of the craniotomy site. The method further includes capturing an overlay image of the craniotomy site and the micro needles using a beamsplitter and an imaging microscope or camera and performing a registration of the overlay image and the craniotomy site based on the coordinates of the micro needles. The method further includes determining implantation sites with the overlay image. The method further includes executing an insertion process of the micro needles, each releasably connected to a flexible neural electrode, into the neural tissue according to an insertion sequence and retracting the micro needles from the neural tissue leaving, for each micro needle, an implanted flexible neural electrode in the neural tissue at a corresponding implantation site of the micro needle.

In one aspect, embodiments disclosed herein relate to a non-transitory machine-readable medium storing a plurality of machine-readable instructions that can be executed by one or more processors associated with a computer-assisted system. The plurality of machine-readable instructions, when executed, cause the one or more processors to perform the following steps. The steps include scanning, with a laser scanner, a craniotomy site with exposed neural tissue and proximate micro needles, forming a laser scan and determining, based on the laser scan, coordinates of the micro needles with respect to a surface of the craniotomy site. The steps further include capturing an overlay image of the craniotomy site and the micro needles using a beamsplitter and an imaging microscope or camera and performing a registration of the overlay image and the craniotomy site based on the coordinates of the micro needles. The steps further include determining implantation sites with the overlay image. The steps further includes executing an insertion process of the micro needles, each releasably connected to a flexible neural electrode, into the neural tissue according to an insertion sequence and retracting the micro needles from the neural tissue leaving, for each micro needle, an implanted flexible neural electrode in the neural tissue at a corresponding implantation site of the micro needle.

In one aspect, embodiments disclosed herein relate to a method for fabricating a micro needle and flexible neural electrode assembly. The method includes temporarily disposing a flexible neural electrode probe including flexible neural electrodes on a jig where the flexible neural electrodes are aligned and held by a substrate. The method further includes aligning micro needles, the micro needles being interconnected or pre-mounted on a needle rack, with distal ends of the flexible neural electrodes of the flexible neural electrode probe. The micro needles whether pre-mounted or interconnected have a defined inter-needle spacing. The method further includes engaging tips of the micro needles in holes at the distal ends of the flexible neural electrodes and picking up the flexible neural electrodes from the substrate.

Other aspects and advantages of the claimed subject matter will be apparent from the following description and the appended claims.

### BRIEF DESCRIPTION OF DRAWINGS

FIGS. 1A and 1B depict a system in accordance with one or more embodiments.

FIG. 2A depicts an insertion robot in accordance with one or more embodiments.

FIGS. 2B and 2C depict a distal portion of an insertion robot in accordance with one or more embodiments.

FIG. 2D depicts a base of an insertion robot in accordance with one or more embodiments.

FIG. 2E depicts an actuator of an insertion robot in accordance with one or more embodiments.

FIGS. 2F-2H depict orthogonal views of holders of an insertion robot in accordance with one or more embodiments.

FIG. 2I depicts manipulator arms of an insertion robot in accordance with one or more embodiments.

FIG. 2J depicts bent tubing in accordance with one or more embodiments.

FIGS. 2K and 2L depict a micro needle in accordance with one or more embodiments.

FIG. 3A depicts beamsplitter and camera system for generating an overlay image of an object such as neural tissue and micro needles in accordance with one or more embodiments.

FIG. 3B an example overlay image in accordance with one or more embodiments.

FIG. 4 depicts a flowchart in accordance with one or more embodiments.

FIG. 5A depicts an example image of a craniotomy site.

FIG. 5B depicts a pattern of micro needles proximate a craniotomy site in accordance with one or more embodiments.

FIG. 5C depicts an example image of a craniotomy site.

FIG. 5D depicts a pattern of micro needles proximate a craniotomy site in accordance with one or more embodiments.

FIG. 6 depicts a flowchart in accordance with one or more embodiments.

FIG. 7A depicts laser-machined micro needles according to a given geometry in accordance with one or more embodiments.

FIG. 7B depicts a nanoelectronic thread (NET) probe in accordance with one or more embodiments.

FIGS. 7C, 7D, and 7E depict a front view, side view, and top view of a needle fixture and needle rack with micro needles in accordance with one or more embodiments.

FIGS. 7F and 7G depict a jig for supporting and aligning a nanoelectronic thread (NET) probe in accordance with one or more embodiments.

FIG. 7H depicts a nanoelectronic thread and micro needle in accordance with one or more embodiments.

FIG. 7I depicts a micro needle and nanoelectronic thread assembly in accordance with one or more embodiments.

FIG. 8 depicts a flowchart in accordance with one or more embodiments.

FIG. 9A depicts a micro needle, nanoelectronic thread, and bent tubing assembly in accordance with one or more embodiments.

FIG. 9B depicts a micro needle, nanoelectronic thread, and bent tubing assembly on a mounting platform in accordance with one or more embodiments.

FIG. 9C depicts an enlarged view of a mounting cap, mounting base, micro needles, and bent tubing in accordance with one or more embodiments.

FIG. 9D depicts a NET probe connected to micro needles supported by a mounting cap and mounting base in accordance with one or more embodiments.

FIG. 9E depicts a mounting base in accordance with one or more embodiments.

FIG. 9F depicts a mounting cap in accordance with one or more embodiments.

FIG. 9G depicts micro grooves of a mounting cap or mounting base in accordance with one or more embodiments.

FIGS. 9H and 9I depict a front and side view, respectively, of micro needles supported by a mounting base and a mounting cap in accordance with one or more embodiments.

FIGS. 9J and 9K depict a threading procedure of micro needles supported by a mounting cap and a mounting base to nanoelectronic threads of a nanoelectronic thread (NET) probe supported by a jig in accordance with one or more embodiments.

FIG. 10 depicts a flowchart in accordance with one or more embodiments.

FIG. 11A depicts interconnected micro needles in accordance with one or more embodiments.

FIG. 11B depicts an enlarged view of interconnected micro needles in accordance with one or more embodiments.

FIG. 11C depicts a post at the distal ends of interconnected micro needles in accordance with one or more embodiments.

FIG. 11D depicts an interconnected micro needle and nanoelectronic thread assembly in accordance with one or more embodiments.

FIGS. 11E-11G depict nanoelectronic threads being picked up by interconnected micro needles in accordance with one or more embodiments.

FIG. 12 depicts a system in accordance with one or more embodiments.

FIG. 13 depicts a graphical user interface in accordance with one or more embodiments.

### DETAILED DESCRIPTION

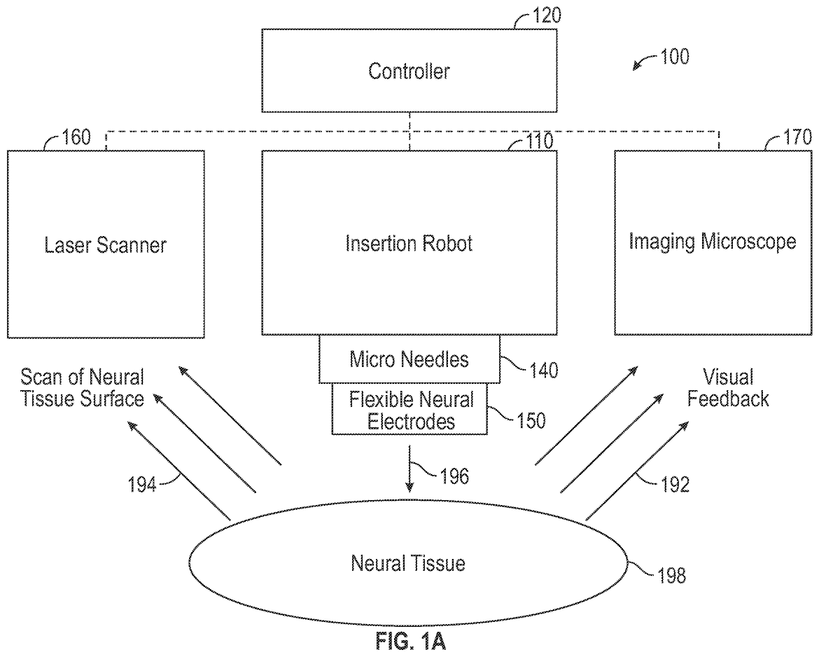

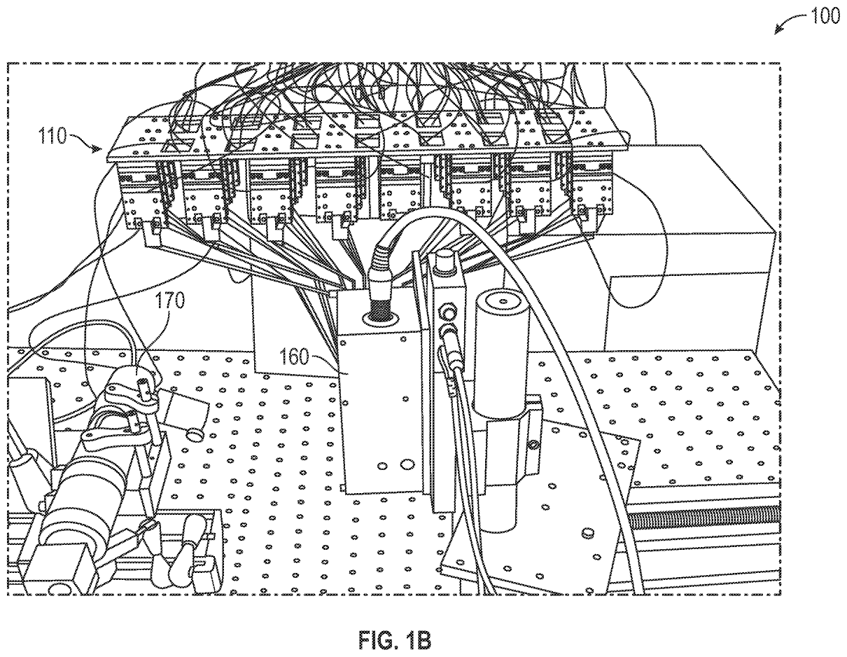

FIG. 1A depicts a diagram of a surgical system (100) for implanting flexible neural electrodes (150) such as nanoelectronic threads (NET). FIG. 1B depicts aspects of the surgical system (100). In general, a chronically implanted flexible neural electrode (150) allows for the monitoring and, in some instances, the stimulation of neuron electrical activities in neural tissue (198). In accordance with one or more embodiments, the surgical system (100) includes an insertion robot (110). As will be described in greater detail later in the instant disclosure, the insertion robot (110) can implant one or more flexible neural electrodes (150) into neural tissue (198) with high spatial accuracy and control. Further, the insertion robot (110) can implant two or more flexible neural electrodes (150) into neural tissue (198) simultaneously, or within a quick sequence, reducing implantation time and mitigating damage of the neural tissue (198). The surgical system (100) further includes micro needles (140) that can be attached to manipulator arms of the insertion robot (110).

In one or more embodiments, and as described below, the insertion robot (110) includes 32 manipulator arms that can be operated independently, in parallel, and with coordinated movements (e.g., to prevent collisions between the manipulator arms and the attached micro needles (140)). Thus, in these embodiments, 32 micro needles (140) can be attached to, and manipulated by, the insertion robot (110). The surgical system (100) further includes one or more flexible neural electrodes (150). A flexible neural electrode allows for electrical communication between the flexible neural electrode and its surrounding environment, e.g., neural tissue. Flexible neural electrodes typically have a small footprint, for example, a nanoelectronic thread (NET) (e.g., that may be considered a type of flexible neural electrode) may have a cross-sectional area or diameter given in microns (e.g., 1 μm×10 μm, 1.5 μm×50 μm). The reduction in dimension and rigidity of flexible neural electrodes compared to conventional neural probes reduces tissue response at the probe-tissue interface, in some instances, a flexible neural electrode (e.g., a NET) can interface with a single cell. Flexible neural electrodes (150) are the recording/stimulation devices that are implanted in the neural tissue (198) (e.g., brain). There are multiple (e.g., 32) flexible neural electrodes in one flexible neural electrode probe/assembly, and multiple (e.g., 16) electrode channels may be integrated on each flexible neural electrode. The flexible neural electrodes, once implanted, may record bioelectric signals from the brain, and/or may deliver electric stimuli to the brain, depending on the specific application requirements.

Various methods for attaching or interfacing flexible neural electrodes (150) with the micro needles (140) are disclosed below. For example, in one or more embodiments, an assembly of flexible neural electrodes (150) attached or adhered to micro needles (140), where there is a one-to-one relationship between flexible neural electrodes (150) and the micro needles (140), is produced according to an assembly method. The flexible neural electrode/needles assembly is a stand-alone package that can be conveniently handled and shipped. The insertion robot (110), through manipulation of micro needles (140) attached to its manipulator arms is used for insertion (196) of the flexible neural electrodes (150) into the neural tissue (198), as depicted in FIG. 1A.

In one or more embodiments, the surgical system (100) further includes a controller (120) or control system. The controller (120) has one or more computer processors and is in electrical communication with, at least, the insertion robot (110). In one or more embodiments, the controller (120) may be considered a component of the insertion robot (110). In one or more embodiments, the controller (120) includes a non-transitory machine-readable medium for storing instructions (e.g., robot software) that when executed by the one or more computer processors cause the insertion robot to perform one or more methods described herein, such coupling, attaching, or adhering the micro needles (140) and the flexible neural electrodes (150) and insertion (196) of the flexible neural electrodes (150) into the neural tissue (198). Thus, a non-transitory machine-readable medium may include a plurality of machine-readable instructions executed by one or more processors associated with a computer-assisted system, the plurality of machine-readable instructions causing the one or more processors to perform the method.

The surgical system (100) can further include a laser scanner (160) and an imaging microscope (170). The laser scanner (160) detects the accurate position of each micro needle before insertion. The imaging microscope (170) provides a live view of the neural tissue (198) surface during insertion (196). In one or more embodiments, one or more laser scanner (160) and the imaging microscope (170) are in electrical communication with the controller (120). As such, manipulation of the insertion robot (110) can be informed by information received from the laser scanner (160) and/or the imaging microscope (170) such as the position (i.e., location and orientation) of the micro needles (140), the position of the neural tissue (198) and the topology of its surface, and the spatial relationship between the micro needles (140) and the neural tissue (198). For example, information received from the laser scanner (160) can include a scan of the neural tissue surface (194). Further, information received from the imaging microscope (170) can include visual feedback (192) or a real-time (or near real-time or traceable time) visual representation of the neural tissue (198) and its environment (e.g., approaching micro needles (140), manipulator arms of the insertion robot (110), etc.). More details surrounding individual components of the surgical system (100) such as the laser scanner (160) and imaging microscope (170) are provided below. In summary, in accordance with at least one method of the present disclosure, the insertion robot engages flexible neural electrode (150) and inserts the flexible neural electrode into neural tissue (198) such as a brain.

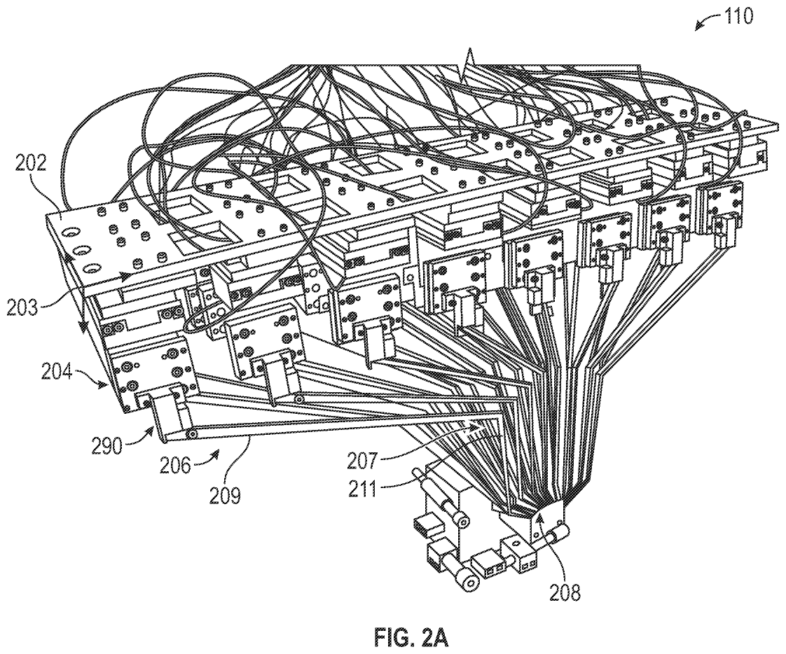

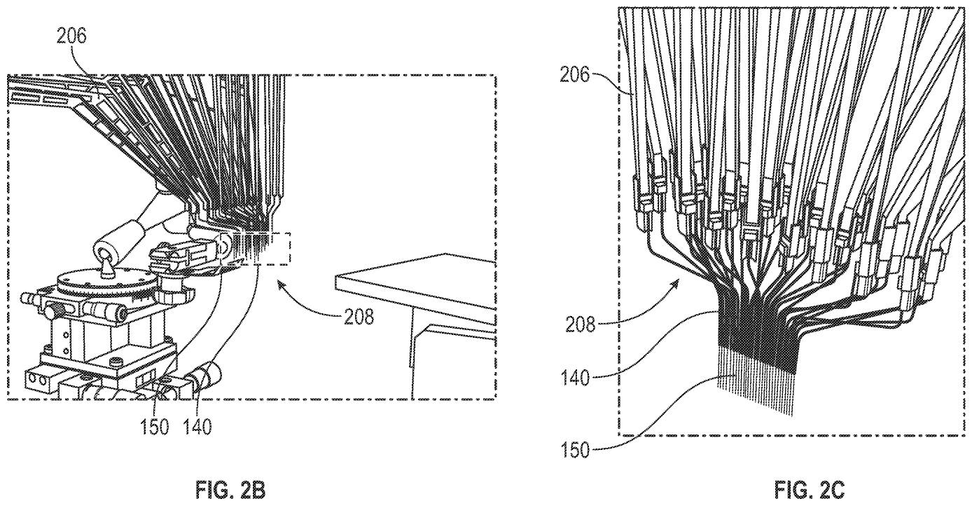

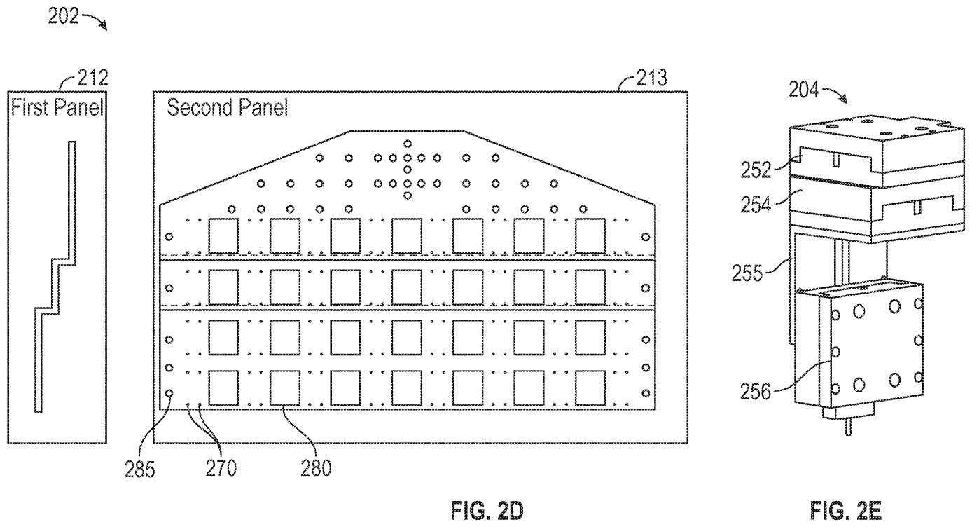

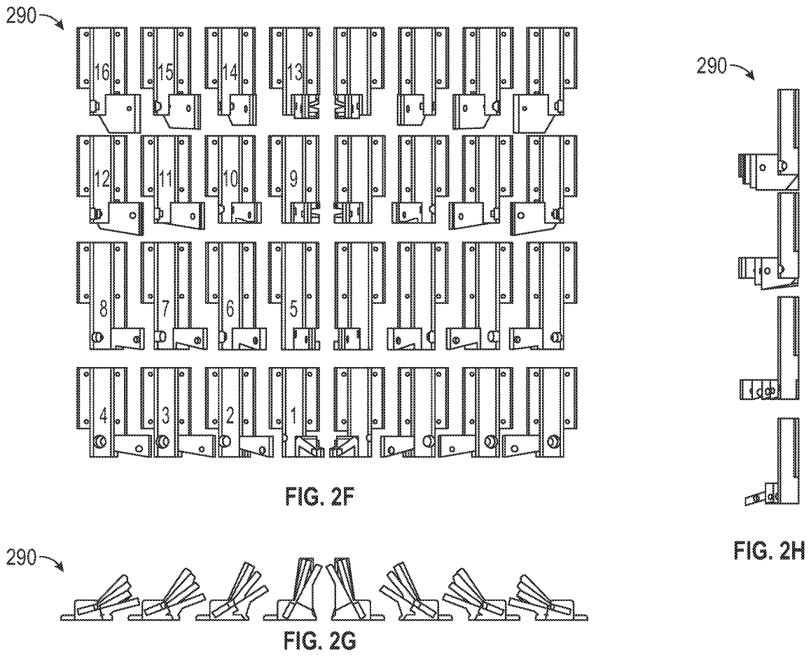

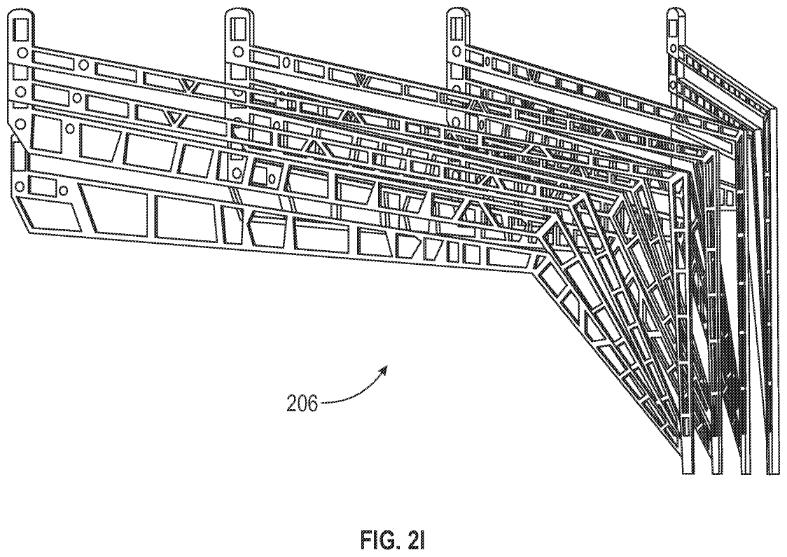

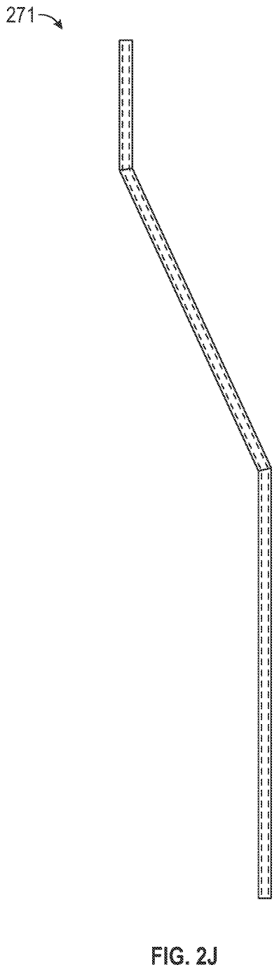

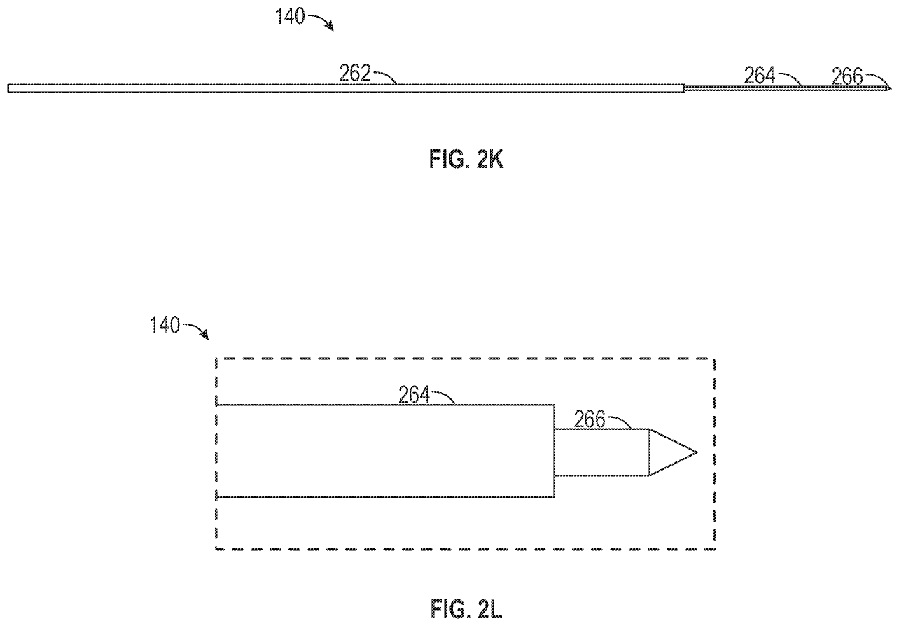

FIG. 2A depicts the insertion robot (110) in accordance with one or more embodiments. As shown, the insertion robot includes a base (202) that supports one or more actuators (204). The base (202) may be affixed to a stationary or mobile structure such as a work bench. As such, the base (202) provides a fixed datum for distal components of the insertion robot (110), where “distal” refers to a direction away from the base along a kinematic series of the insertion robot (110) toward an end effector (208) ultimately manipulated using the insertion robot (110). Similarly, a “proximal” component is disposed nearer to the base (202). In some embodiments, the base (202) is supported by a vertical stage (not depicted) such that the base (202) may be translated in a vertical direction, e.g., along an axis orthogonal to a horizontal plane formed by a work bench. As discussed, the insertion robot (110) may be used to implant flexible neural electrodes (150) into neural tissue (198), e.g., a brain.

The insertion robot (110) includes multiple manipulator arms (206), e.g., 32 manipulator arms, each of which can be independently controlled. As depicted in FIG. 2A, a distal portion of each manipulator arm is coupled to an actuator (204) supported by the base (202). Specifically, there is an actuator (204) for each manipulator arm (206). As such, independent control of each manipulator arm (206) is effectuated using, at least in part, its associated actuator (204). In one or more embodiments, an actuator (204) is composed of one or more linear stages stacked in series to provide linear movement in multiple directions. Further, in some embodiments, each manipulator arm (206) is attached to an actuator (204) using an arm junction (290). More details surrounding base (202), actuators (204) (or, at least a linear stage of the actuator), arm junctions (290), manipulator arms (206), and other items such a bent tubing and the micro needles (140) are provided below with respect to FIGS. 2D-2J.

In one or more embodiments, the actuators (204) are arranged on the base (202) according to a specified arrangement. For example, FIG. 2A depicts an instance where the actuators (204) are arranged on the base (202) to form a 4 by 8 grid (i.e., 32 actuators in total). That is, in FIG. 2A, the specified arrangement may be said to be a 4 by 8 grid. Specifically, the grid is given according to a grid spacing with respect to a plane coplanar with a surface of the base (202). For example, FIG. 2A depicts a reference coordinate system with three orthogonal axes, namely, an x axis, a y axis, and a z axis. The x axis and the y axis form a so-called XY plane that is coplanar with a surface of the base (202). Thus, spacing between any pair of actuators, e.g., a first actuator and a second actuator, in the x direction can be specified as Δx1,2 and spacing between that pair of actuators in the y direction can be specified as Δy1,2, where the subscripts 1 and 2 refer to the first and second actuators in the pair of actuators, respectively. In some embodiments, such as that depicted in FIG. 2A, the spacing between adjacent actuators along the x axis is the same. That is, the actuators are equidistantly spaced in the x direction. Similarly, the spacing between adjacent actuators along the y axis is the same, however, the spacing between the adjacent actuators need not be the same in the x and y directions. Further, the spacing, whether in the x or y directions, need not be equal for all pairs of adjacent actuators. That is, the actuators (204) can be arranged non-uniformly. While FIG. 2A depicts the actuators (204) arranged to form a grid, this need not be the case. Other arrangements of the actuators (204) on the base can be used without departing from the scope of this disclosure. As an example, the actuators (204) can be arranged on the base (202) as a linear array. Further, different grid arrangements can be used without departing from the scope of this disclosure. For example, using 32 actuators (204), the actuators (204) can be arranged on the base (202) to form a 2 by 16 grid. In general, the arrangement of the actuators (204) with respect to the base (202) is specified by one or more arrangement parameters, where the arrangement parameters are said to contain all necessary information to describe the arrangement (e.g., grid layout, grid spacing, coordinate locations of each actuator, etc.).

Given the arrangement of the actuators (204), and the size of each actuator, a first area spanned by the actuators (204) can be determined. Further, in some embodiments, the height (or length of an actuator in the vertical direction) is also considered to determine a first volume spanned by the actuators (204).

In one or more embodiments, manipulator arms (206) include one or more bends. Further, a bend in a manipulator arm (206) can partition the bend into various portions or links. Portions or links of a manipulator arm (206) bounding a bend need not have the same length or profile. In greater detail, in FIG. 2A, a manipulator arm (206) is depicted as having a first bend (207) that partitions the manipulator arm (206) into a first portion (209) and a second portion (211). In general, a manipulator arm (206) can have more than one bend (e.g., a first bend and a send bend) and any number of portions or links between respective bends. Further, various manipulator arms (206) of the insertion robot (110) can have different lengths of first portions (209), different lengths of second portions (211), and different bend angles for their respective first bends (207). In accordance with one or more embodiments, the cross-sectional area of a manipulator arm steadily decreases from the proximal end of the manipulator arm (206) to its distal end.

This distal end of a manipulator arm (206) is configured with, or can interact with, various end effectors (208). As will be described below, in some embodiments, the end effectors (208) are micro needles (140) (e.g., laser-machined micro needles). In other embodiments, each of the end effectors (208) is a bent tubing where a micro needle (e.g., laser machined tungsten wire) can be attached or otherwise affixed to the distal end of the end effector.

FIGS. 2B and 2C depict the distal ends of manipulator arms (206) of the insertion robot (110) in greater detail, in accordance with one or more embodiments. In the depiction of the FIGS. 2B and 2C, the end effector (208) supported by each manipulator arm (206) is a bent tubing. The bent tubing is further configured to connect to, or otherwise hold, a micro needle (140) (e.g., laser-machined tungsten wire). Further, each micro needle (140) threads or releasably attaches to one flexible neural electrode (150).

In some embodiments, the distal portion of the manipulator arm (206) or its end effectors (208) can include an articulable portion such as a wrist assembly to provide additional degrees of freedom (e.g., pitch, yaw, and roll). Further, in some embodiments, the articulable portion facilitates the attachment of an end effector (e.g., micro needle). For example, the articulable portion and/or a distal end of the manipulator arms (206) can contain a magnet, be magnetized, or otherwise produce a magnetic force for bounding or attaching the end effectors (e.g., micro needles). Regardless of the type(s) of micro needle (140) employed for use with the insertion robot (110), each micro needle (140) is configured to thread or be releasably connected to one flexible neural electrode (150) (e.g., one flexible electrode shank).

In one or more embodiments, each actuator (204) actuates, or can provide a displacement over a given range of motion, in the x, y, and z directions (i.e., along the x axis, y axis, and z axis of the reference coordinate system (203). That is, each actuator (204) provides three translation degrees of freedom. Due to the rigid connection of a micro needle (140) to an actuator (204) through a manipulator arm (206), each micro needle (140) may move independently in, at least, the x, y, and z directions. That is, the insertion robot (110) has a one-to-one-to-one relationship between actuators (204), manipulator arms (206) (and end effectors (208)), and micro needles (140) such that each micro needle (140) can be driven in the x, y and z directions by its corresponding actuator (204) supported by the base (202).

In general, actuation of a micro needle (140) by its corresponding actuator (204), and thus a threaded or releasably connected flexible neural electrode (150) during operation of the insertion robot (110), in the x direction and the y direction provide for adjustment of insertion sites according to operator's (e.g., surgeon) request. Further, actuation of a micro needle (140) by its corresponding actuator (204) in the z direction executes insertion and retraction operations. The actuators (204) may be piezoelectrical motors or any other types of actuators with micrometer precision of movements.

In other embodiments, one or more actuators (204) provide additional degrees of freedom, e.g., rotation about one or more of the x, y and z axes or pitch, roll, and yaw degrees of freedom with respect to the reference coordinate system (203). In some embodiments, these additional degrees of freedom are provided by the distal end or end effector (208) of the manipulator arms (206), e.g., using an articulable wrist assembly. A benefit of these additional degrees of freedom, whether provided by the actuator (204) or the manipulator arm (206) (or end effector (208)) is that a micro needle (140) can be oriented relative to the neural tissue (198), e.g., a longitudinal axis of the micro needle (140) can be oriented locally orthogonal to the neural tissue (198).

In accordance with one or more embodiments, the manipulator arms (206) of the insertion robot (110) are configured to reduce the working area or volume of the micro needles (140). As seen in FIGS. 2B and 2C, the micro needles (140) span a second area or a second volume (if considering a length characterizing the micro needles). By virtue of the manipulator arms (206), as described (e.g., having a first bend (207), gradually decreasing cross-sectional area, etc.), the second area or second volume is less than the first area or first volume spanned by the actuators (204). That is, the insertion robot (110) is able to execute a task in a workspace with a smaller spatial footprint than its actuators (204), even with the actuators (204) providing the movement or actuation associated with the executed task. Further, the manipulator arms (206) of the insertion robot (110) are configured such that specified patterns, e.g., a first pattern, a second, pattern, etc., can be formed by the micro needles (140). As an example, a first pattern can be a grid (e.g., 4 by 8 grid) of micro needles (140) and a second pattern can be a linear array of micro needles (140). A pattern may be selected or commanded by an operator and/or by the controller (120) based on a given task. Further, one or more patterns can be realized regardless of the arrangement of the actuators (204) on the base (202). For example, in one or more embodiments, the actuators (204) are arranged according to a grid and the micro needles (140), through actuation of the actuators (204) and due to the design of the manipulator arms (206), can be placed in either a grid (e.g., similar to the arrangement of actuators but at a smaller scale) or in a linear array. That is, although the actuators (204) may be arranged according to a grid the micro needles (140) can have a distinct pattern, relative to the actuator arrangement, such as a linear array.

FIG. 2D depicts a side view and a top view of the base (202) of the insertion robot (110) in a first panel (212) and a second panel (213), respectively. In one or more embodiments, the base (202) is designed to hold 96 piezoelectric motors and enable their maximum range of motion while preventing any collision of the motors between themselves.

The piezoelectric actuators responsible for translation on the horizontal plane are attached to the base plate. As seen in the second panel (213), the base plate has through holes (270) matching motors' threaded holes (M2 size) to tightly secure the motors to the plate. Note, to avoid cluttering the figure only two through holes (270) are labelled in FIG. 2D. Each motor is spaced in the horizontal plane by the maximum travel range of the actuator plus 1 mm to allow room for the cable, making it impossible for two motors to collide in this direction. The same principle was applied to the longitudinal plane. Thus, the minimal spacing between the motors that prevents collisions in those two directions was achieved.

The base plate has square holes (280) next to each azimuth actuator to allow the cables and plugs of the motors to be secured above the base plate, preventing cable interferences with surgical arms motion. Again, to avoid cluttering the figure, only one square hole (280) is labelled.

As seen in the first panel (212), the base (202) has a stepped cross section to avoid collision of surgical arms along the vertical axis. The motors on the third row are lowered compared to the motors of the first two rows, and the motors from the fourth row are lowered compared to the motors of the third row.

In one or more embodiments, the base (202) is attached to a support pillar from the back of the plate, behind the fourth row. As discussed above, the base (202) elevation or vertical height with respect to horizontal surface such as a work bench can be adjusted on the pillar to satisfy surgical procedure needs. In some embodiments, an angle of the base (202) with respect to the support pillar (or work surface) can be adjusted. For example, the attachment of the base (202) to the support pillar can include an articulable portion allowing for rotations (e.g., pitch, yaw, roll) of the base (202) relative to the support pillar.

In some embodiments, additional holes (285) on the edge of the base (202) have been added to attach different devices necessary to surgical procedures (e.g., camera, light, lasers, tubes, additional motors, etc.)

The base (202) can be made of various metals that would not deform under the weight of the manipulator arms (206) such as titanium, stainless steel, and aluminum, among other materials not listed.

In one or more embodiments, compact size motors reaching high acceleration and slow constant speed are used in the insertion robot. In one or more embodiments, the motors are compact piezoelectrical motors. Piezoelectrical motors enable slow speed insertion (5 μm/s) for scarless insertion of flexible neural electrodes attached to rigid micro needles (this slow insertion speed significantly reduces tissue damages). Fast insertions have been shown to create significant damage to neural tissues, hindering recording and stimulation of nervous cells. Herein insertion speeds are achieved that result in scarless insertion of flexible neural electrodes.

To deliver the flexible neural electrodes into the neural tissue, the micro needles need to be retracted with high acceleration to avoid also retracting the flexible neural electrodes or creating tissue damage during upward movement (from undesired lateral movements). The piezo motors used here achieve higher accelerations than previously used technology (e.g., greater than 30 m/s2).

Motors are mounted in bundles of three to allow for horizontal, longitudinal, and vertical movements of the manipulator arms during all steps necessary to surgical implantation of flexible neural electrodes. FIG. 2E depicts the stacking of motors to form an actuator (204), in accordance with one or more embodiments. As seen, the actuator (204) consist of a first motor or first stage (252), a second motor or second stage (254), and a third motor or third stage (256) to provide movement of a supported manipulator arm (206) in three orthogonal directions (e.g., along the x axis, y axis, and z axis). To connect stages moving horizontally and longitudinally (connection of stages 252, 254), existing holes threaded by the motors' manufacturer were used. To connect stages moving longitudinally and vertically (connection of stages 254, 256), a custom L-bracket (255) matching the manufacturer's holes was invented and machined from aluminum.

In one or more embodiments, to translate movement of the actuators (204) to surgical insertion site, the manipulator arms (206) are attached to the third stage (256) using junctions referred to herein as “holders” or “arm junctions” (290).

In one or more embodiments employing an insertion robot that supports 32 manipulator arms, there are 16 unique holders that are mirrored to attach the 32 manipulator arms to the vertical stages (256). Each holder has a very specific and unique angle with the horizontal plane, so that the tips of the 32 manipulator arms form a 4 by 8 array covering 21 by 49 mm² (spacing between each tip being 2 mm in both the horizontal and longitudinal planes). Offering a total coverage for surgical implantation of 39 by 67 mm² (considering each motor has an 18 mm range of motion and applying it to horizontal and longitudinal movements).

Each holder has been designed to maximize the contact area at the interface with the vertical motor as well as at the interface with the arm. By maximizing the contact area, potential vibrations are reduced during movement of the manipulator arms that could be at the origin of tissue damage. FIGS. 2F, 2G, and 2H depict a front, top, and side view, respectively, of the 16 unique holders (290) including their mirrored counterparts.

Holders (290) can be made using SLA or FDM 3D printing, injection molding, or Direct Metal Laser Sintering or other standard manufacturing techniques.

The angles of the holders (290) and length of the manipulator arms (206) can be modified to enlarge or reduce the coverage of the manipulator arms (206) tip array (i.e., the second area), for applications where the coverage needs to be different than the default 21 by 49 mm² coverage (e.g., surgeries in humans, non-human primates, or pigs).

The 32 holders (290) are attached to 32 manipulator arms (206) to translate actuator (204) movements to the insertion site. There are 16 unique manipulator arms (206) that are produced twice for simultaneous insertion of up to 32 flexible neural electrodes. FIG. 2I depicts various manipulator arms (206) in accordance with one or more embodiments.

The manipulator arms (206) are designed to bring as much verticality as possible, which prevents collisions in the horizontal and longitudinal planes of arms during preparation and insertion of flexible neural electrodes. Additionally, the manipulator arms (206) are designed to not collide with the motor/holder of the row in front of them during vertical movements.

In particular, and has seen in FIG. 2I, each manipulator arm (206) has an inflexion point that lowers (in the vertical direction) as the distance between the insertion site (array formed by the arms' tips or distal ends) and the motor on which the arm is attached increases. In other words, the manipulator arm attached to the holder #1 will have the highest inflexion point and the manipulator arm attached to the holder #16 will have the lowest inflexion point. In this way each manipulator arm (206) can more easily slide under the manipulator arms (206) closer to the insertion site (meaning in front in the longitudinal direction or on its right on the horizontal direction) without collisions when moving manipulator arms (206) before insertion of flexible neural electrodes.

In some embodiments, the manipulator arms (206) are ribbed, or otherwise have material “carved out,” to reduce the weight of the manipulator arms (206) and enable fast retraction of the manipulator arms (206) after implantation of the flexible neural electrodes. As stated, it is important that the manipulator arms (206) retract with a high acceleration to ensure they do not pull out the flexible neural electrodes or damage tissue with lateral movements in the process. In one or more embodiments, the manipulator arms (206) are designed using finite element analysis (FEA) to remove material without sacrificing stiffness of the manipulator arm (206).

The manipulator arms (206) can be made of any biocompatible material that is stiff enough for this application, examples include: titanium, stainless steel, aluminum or other biocompatible metals, biocompatible 3D printed resins, and biocompatible plastics. In one or more embodiments, the manipulator arms (206) are manufactured using standard computer numerical control (CNC) machining tools (e.g., waterjet, laser cutter, mill, etc.) as well as plastic injection molding or other conventional machining technique that would be appropriate.

In one or more embodiments, the manipulator arms (206) support, or are otherwise attached to, bent tubing (271). A representative bent tubing (271) is depicted in FIG. 2J. The bent tubings (271) serve as connective structures between the micro needles (140) and the manipulator arms (206). In one or more embodiments, they are fabricated from hypodermic tubings. In one or more embodiments, they feature a 300-um outer diameter (OD) and a 150-μm inner diameter (ID). The proximal or rear ends of micro needles (140) are inserted into the bent tubing's (271) inner wall and fixed by superglue. The proximal or upper part of the bent tubing (271) is then fixed on the manipulator arms (206) by superglue.

The design of one micro needle is depicted in FIGS. 2K and 2L, in accordance with one or more embodiments. Specifically, FIG. 2K is a side view of the micro needle (140) and FIG. 2L is an enlarged view of the distal end of the micro needle (140). As depicted in FIGS. 2K and 2L, the micro needle (140) has a cylindrical structure with different diameters for different portions. In some embodiments, the bulk shaft (262) has 100 μm diameter and can fit into the inner diameter of the bent tubings (271). The bulk shaft with larger diameter ensures that the micro needle is strong enough during insertion (i.e., the micro needle won't buckle). To minimize tissue damage, the bulk shaft will not go into the neural tissue during implantation. The insertion shaft (264) can have a diameter between 20 μm and 100 μm and will go into the neural tissue during implantation. The micro tip (266) is used for mechanically anchoring (or threading) with a flexible neural electrode, thus being able to drive the flexible neural electrode into neural tissue.

In review, with 32 manipulator arms (206) working together, 32 flexible neural electrodes can be inserted into neural tissue (198) (e.g., brain), either simultaneously or sequentially. Each insertion site can be flexibly designated by an operator (e.g., surgeon and/or researcher) and can be targeted with micrometer precision and an flexible neural electrode can be inserted with speeds ranging from 1 μm/s to 200 mm/s. Returning to FIG. 1A, the controller (120) may be used to control calibration and operation of the insertion robot (110) during surgeries in accordance with embodiments of the disclosure. The controller (120) executes instructions or software including various functionalities such as, but not limited to: initialization of the surgical system (100) and communication with the laser scanner (160). The controller (120) can further receive and respond to feedback of 1. Sensor signals from the actuators (position, speed, etc.) and 2. Visual signals (camera or microscope, e.g., imaging microscope (170)), preoperative planning (shapes for surgical insertions), and performing surgical procedures (single actuator control, threading neural probes, probes implantation and retraction, etc.).

The laser scanner (160) serves as a position sensor, in accordance with embodiments of the disclosure. The laser scanner (160) to find the real position of the micro needles. The motors have a function to find their real space position, but this function is not always reliable. The laser scanner (160) is used to find the micro needle positions in three dimensions with tens of microns precision. The laser scanner (160) is used in coordination with an algorithm that automatically identifies the tip or distal end of each micro needle or wire in a three-dimensional point cloud file returned from the laser scanner (160). The algorithm then moves some micro needles or wires in each direction (longitudinally, horizontally, and vertically, or along the x, y, and z axes) by a known distance and finally performs a second scan to automatically identify the tip or distal end of the micro needles or wires again. Using the tips coordinates from the first and second scans, and the distances each the wire has moved between the first and second scans, the controller (or processing unit described below) calculates the transformation matrix linking the scanner's longitudinal, horizontal, and vertical axes to the micro needles longitudinal, horizontal, and vertical axes. The controller (or processing unit described below) then applies this matrix to the point cloud of the second scan (reflecting the current positions of the micro needles or wires), to obtain a matrix of tips positions that is used to update the real space positions of the wires on a three-dimensional representation that can be displayed using a graphical user interface (GUI) or other display.

Further, the laser scanner (160) scans the micro needles (140) and neural tissue (198) surface for calibration and surgery planning and returns the XYZ coordinates (i.e., an x, y, and z value according to a specified cartesian coordinate frame such as reference coordinate system (203)) of each micro needle (140) relative to the neural tissue surface. These coordinates may then be used to calculate the distances and directions by which each micro needle (140) needs to move, to reach the desired implantation placement of each flexible neural electrode designated by the operator. In some embodiments, a registration of an image obtained by the imaging microscope (170) and the coordinates of the micro needles (140) is performed. This process is explained in greater detail below. However, with this registration, determination of implantation placement of each flexible neural electrode can be performed automatically by the controller (120) and, for example, monitored by an operator.

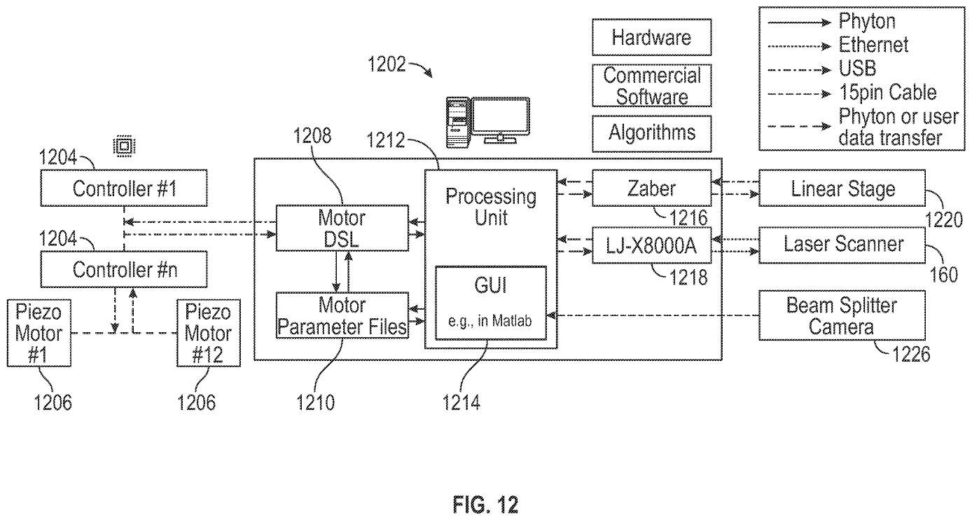

In one or more embodiments, the laser scanner (160) is attached to a laser linear stage for movement in the vertical direction to obtain a full three-dimensional profile of the micro needles (i.e., the laser scans for depth in the horizontal plane, and it is moved at consistent speed in the vertical plane to obtain a three-dimensional profile). The laser linear stage is controlled by Zaber Software (depicted in FIG. 12) but also has a domain specific language (DSL) that can be interfaced using Python so the laser linear stage can be controlled by the controller (120).

The imaging microscope (170) provides live visual feedback for manual manipulations and process monitoring, in accordance with embodiments of the disclosure. The operator may use the microscope when threading the micro needles (140) with the flexible neural electrodes (150). The operator may use the imaging microscope (170) to monitor the insertion and retraction process.

In some embodiments, a beamsplitter can be mounted to the imaging microscope (170) (or camera) coaxially and between the flexible neural electrodes (150) and neural tissue (198) to get an overlayed image of the two views for aligning the flexible neural electrode to the desired implantation sites (i.e., registration of the micro needle locations and the neural tissue). This is particularly useful in instances where a top view of the neural tissue (198) surface is not accessible.

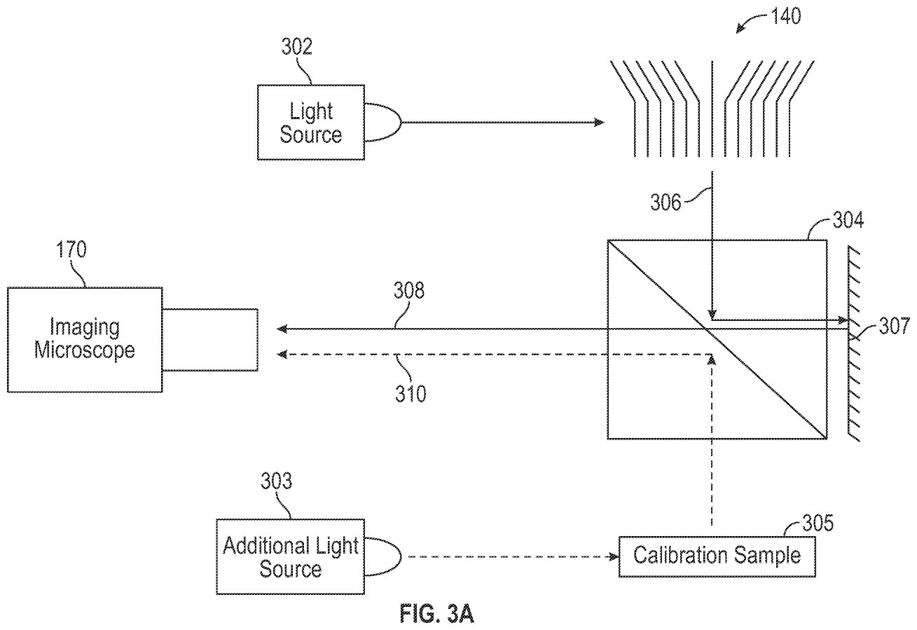

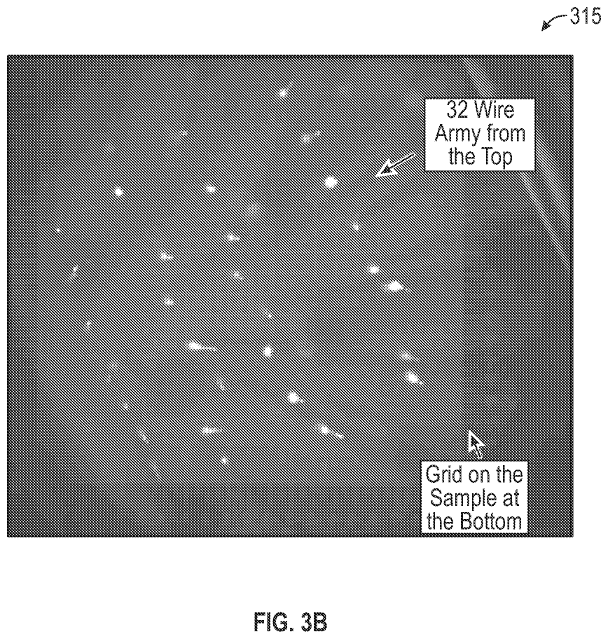

FIG. 3A depicts the use of a beamsplitter (304) to produce an overlaid image of the micro needles (140) (above, or “at the top,” of the beamsplitter) and a calibration sample (305) (below, or “at the bottom,” of the beamsplitter). As will be depicted in FIG. 3B, a calibration sample (305) can be used to determine the relative spacing and locations of micro needles (140) within an image of the imaging microscope (170) (in some instances, a camera can be used in place of the imaging microscope).

In accordance with one or more embodiments, the beamsplitter is temporarily placed between the micro needles (140) (the micro needles (140) being supported by the insertion robot (110)) and the calibration sample (305). As an example, the calibration sample (305) can be a printed two-dimensional grid with a known line spacing and size. An image (310) of the calibration sample (305) is produced at the imaging microscope (170) (or a camera) via a reflection of the calibration sample (305) using the beamsplitter (304). As seen in FIG. 3A, a light source (302) illuminates the micro needles (140). The light source (302) is configured (e.g., selection of wavelength in view of the beamsplitter) such that light (306) emanating from the illuminated micro needles (140) is reflected in the beamsplitter (304) toward a mirror (307) and then reflected back from the mirror (307) as reflected light (308) along a nominal axis back to the imaging microscope (170) to be seen in the image (310) of the calibration sample (305). In some embodiments, an additional light source (303) is used to illuminate the calibration sample (305). With fine control of lighting using one or more of the light source (302) and the additional light source (303) and a configured exposure condition, a clear overlay image (315) is produced, the clear overlay image (315) showing both the micro needles (140) and the calibration sample (305). Thus, positions of the micro needles (140) in the image (310) can be determined in view of the calibration sample (305). An example of an overlay image (315) is provided in FIG. 3B. As seen, a calibration sample (305) is imaged and a grid pattern of micro needles (140) is seen or overlaid in the image.

Once calibrated, the calibration sample (305) is removed and replaced with neural tissue (198). Using an image of the neural tissue (198) and overlaid micro needles (140), implantation sites can be determined (e.g., selected by an operator, determined by the controller, for example, by detection and avoidance of blood vessels). Further, using the image with determined implantation sites and the overlaid positions of the micro needles (140), planar distances between the micro needles (140) and implantation sites can be determined. Then, the beam splitter can be removed and the insertion robot (110) can perform insertion of flexible neural electrodes (150) to the implantation sites guided by the determined planar distances.

A method for operating the previously described system is described below. The method includes two stages; namely, stage 1 and stage 2. In stage 1 an anchoring or releasable connection between flexible neural electrodes and micro needles is established. In stage 2, the flexible neural electrodes are implanted using the surgical system.

In stage 1 the flexible neural electrode and needle assembly is fabricated. Process A or process B may be used. A 32-needle system is assumed for illustration.

### Stage 1A—Process A of Fabricating Needle/Thread Assembly

At stage 1A.1, to initialize the system, the 32 micro needles are pre-mounted on the tips of the 32 robot arms, and are arranged in an ordered format, such as a 4×8 matrix with 2 mm spacings.

At stage 1A.2, the laser scanner is used to acquire the accurate XYZ coordinates of each needle.

At stage 1A.3, the software controls the XYZ actuators to move the micro needles to form a format that can easily match the arrangement of the flexible neural electrode assembly, such as a 1×32 array with 0.5-mm spacings.

At stage 1A.4, the 32 micro needles engage with the 32 flexible neural electrodes and peel the flexible neural electrodes off from the thread holder.

### Stage 1B—Process B of Fabricating Needle/Thread Assembly

At stage1B.1, the 32 micro needles are pre-mounted on a needle rack with defined inter-needle spacing, e.g. 500 μm.

At stage 1B.2, the 32-shank flexible neural electrode probe is released and temporarily fixed on a jig and all flexible neural electrodes are aligned and held by a flat substrate.

At stage 1B.3, the 32 shanks are aligned with the 32 micro needles. The tips of microneedles are engaged in the holes at the tip of each flexible neural electrode.

At stage 1B.4, the microneedles are controlled to move forward and pick up the flexible neural electrodes from the holder substrate.

At stage 1B.5, a dissolvable bio-adhesive is applied to secure the flexible neural electrodes to the microneedles.

In this process, the flexible neural electrode/needles assembly is a stand-alone package that can be conveniently handled and shipped.

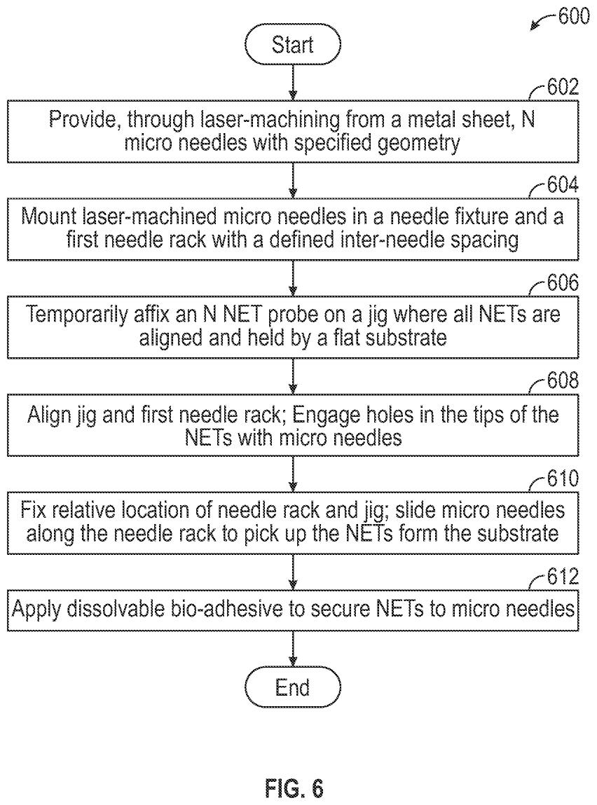

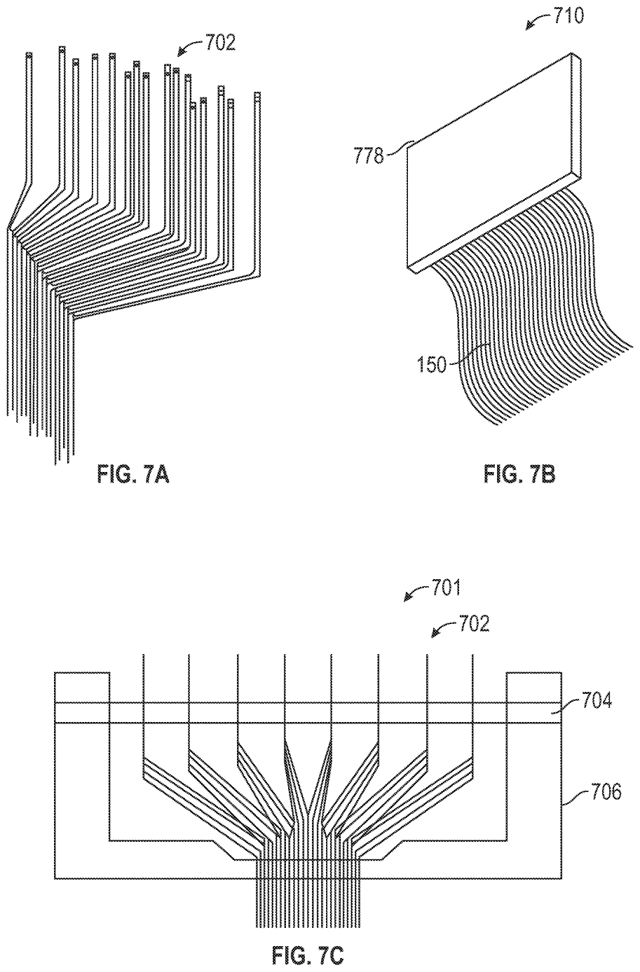

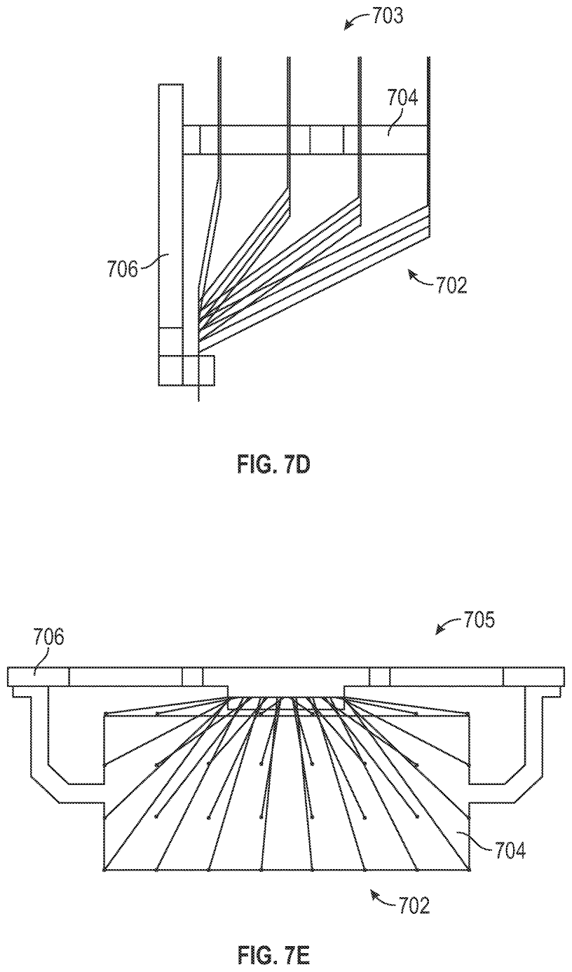

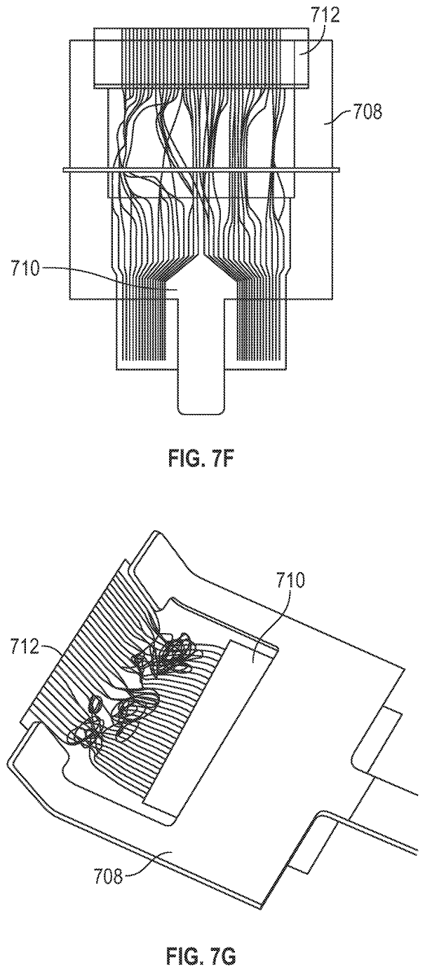

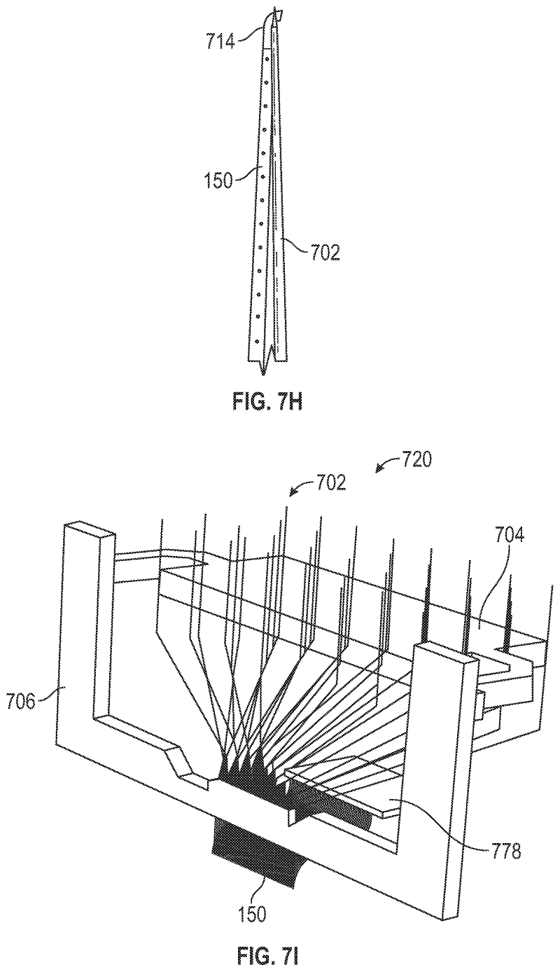

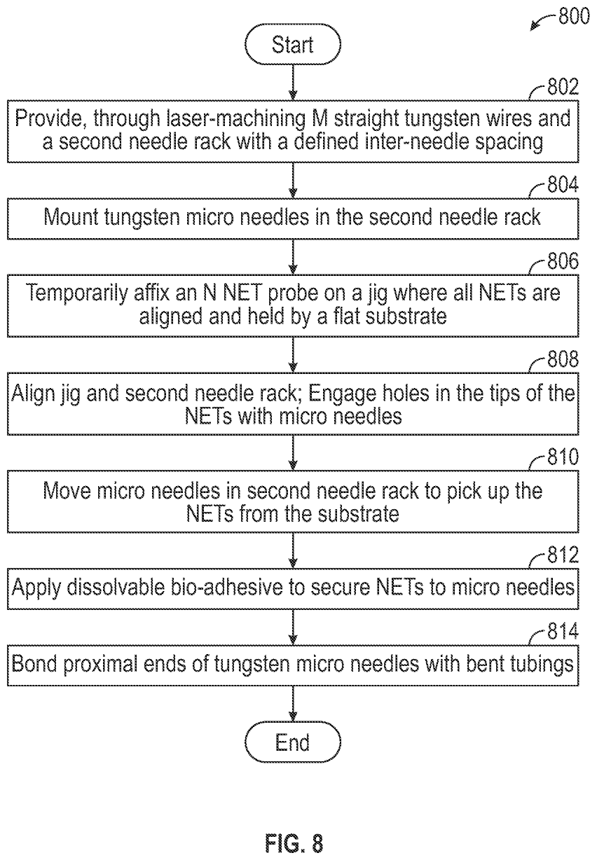

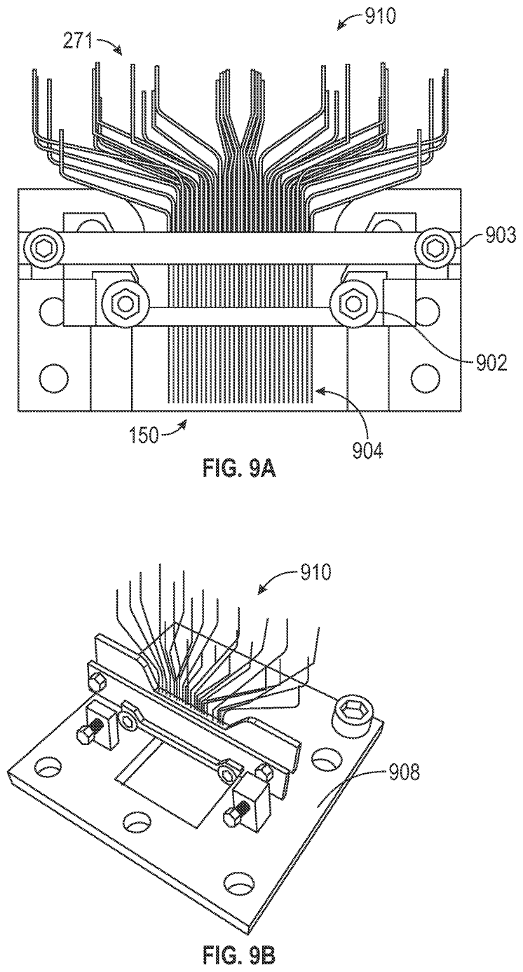

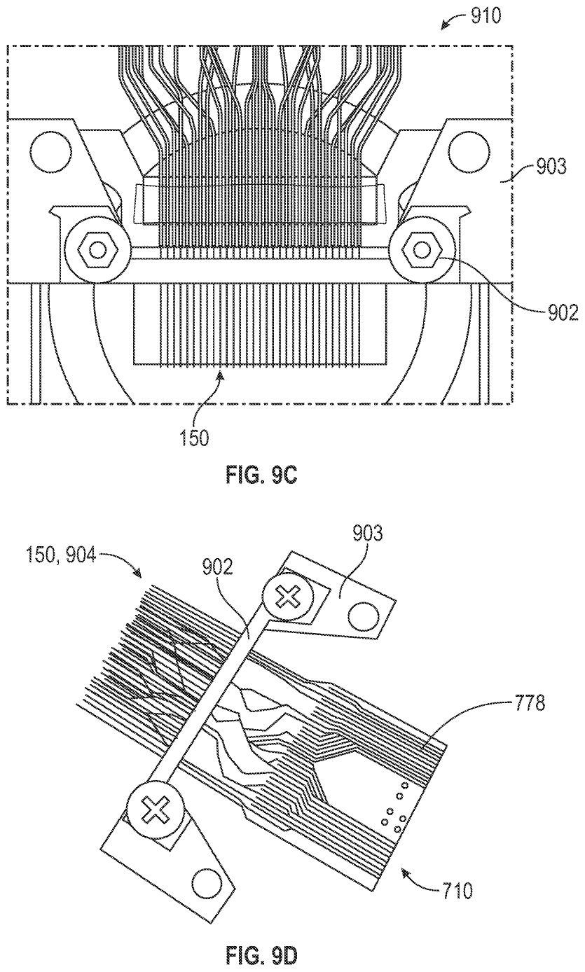

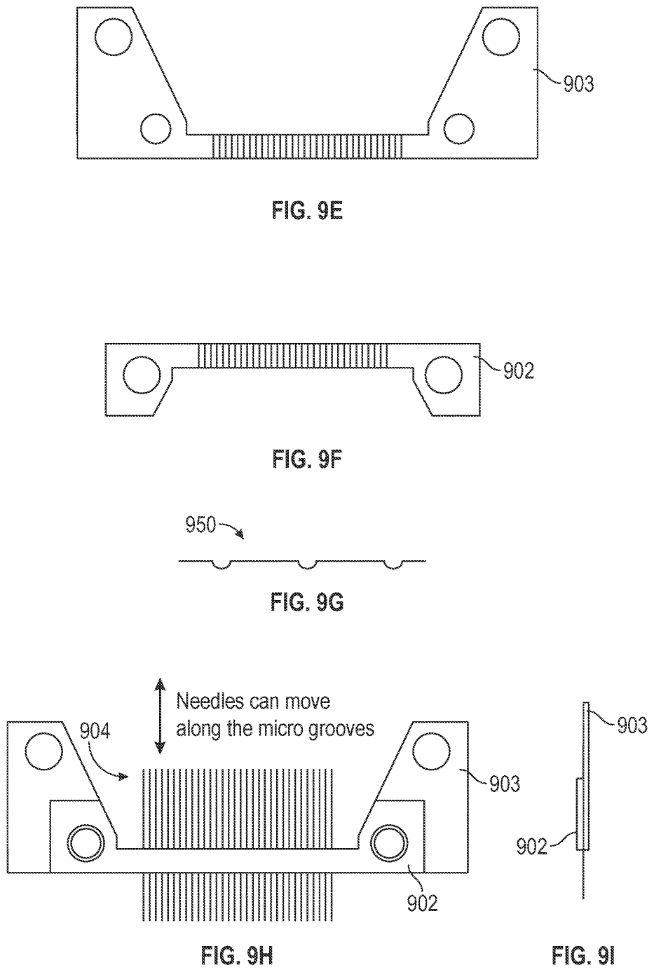

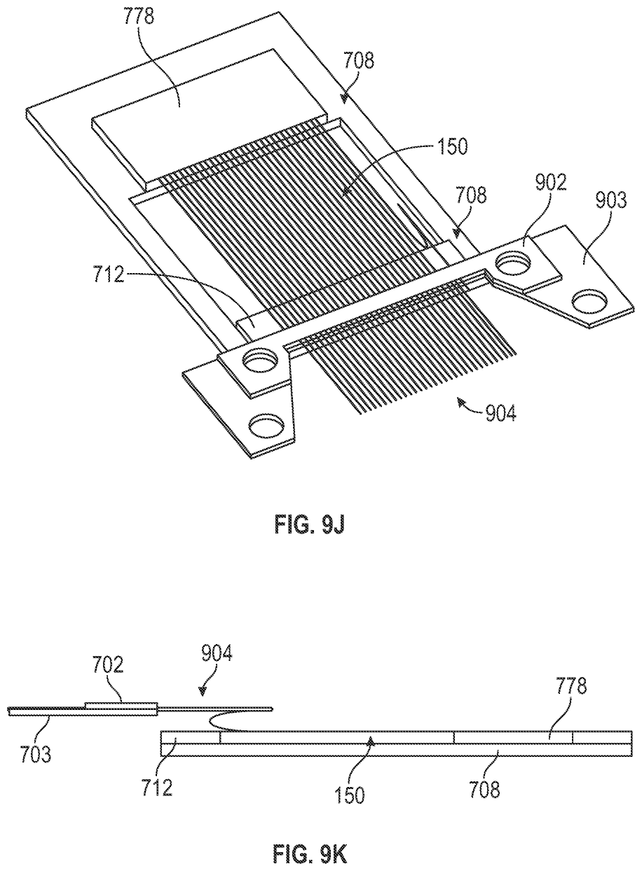

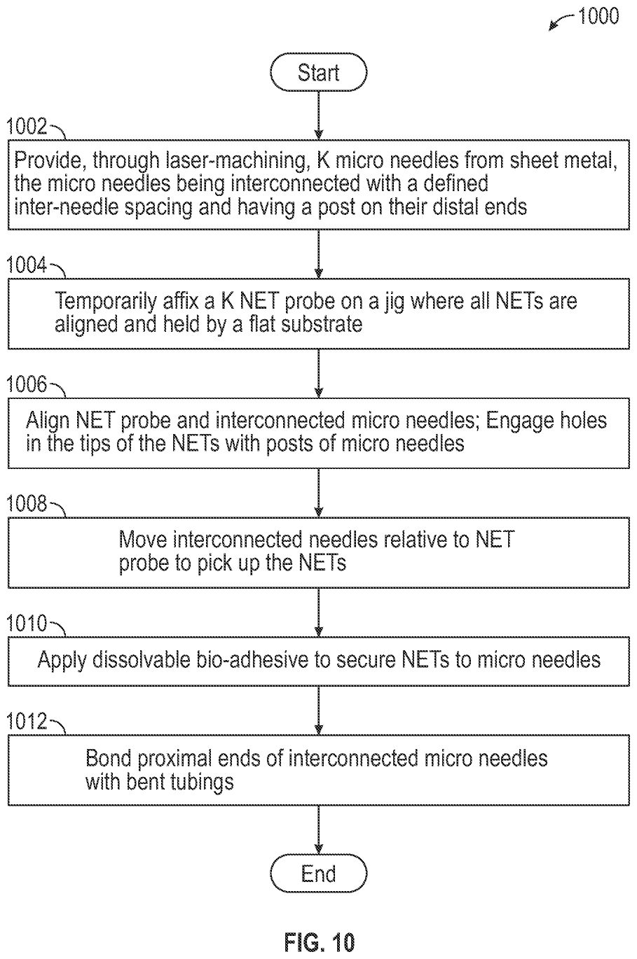

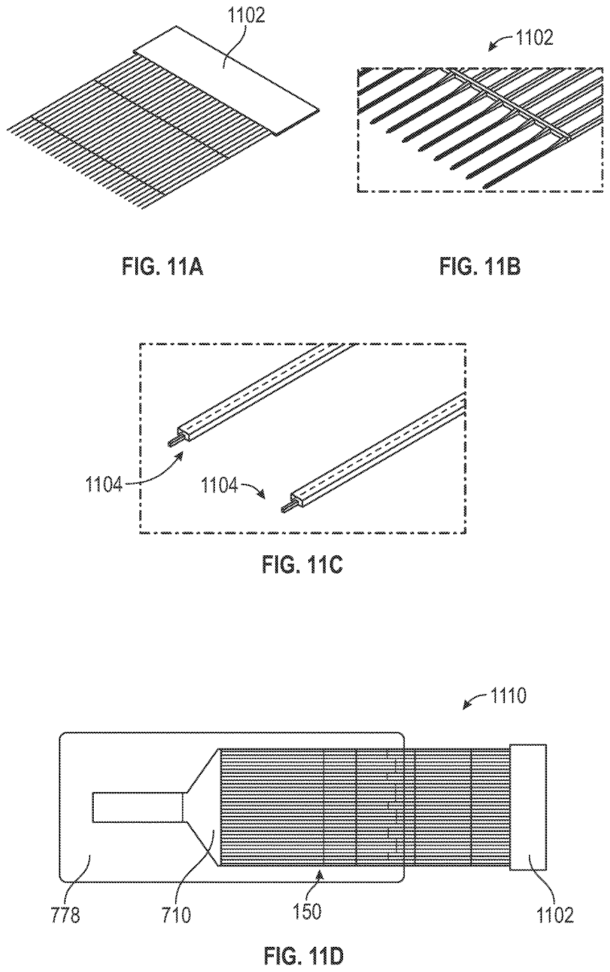

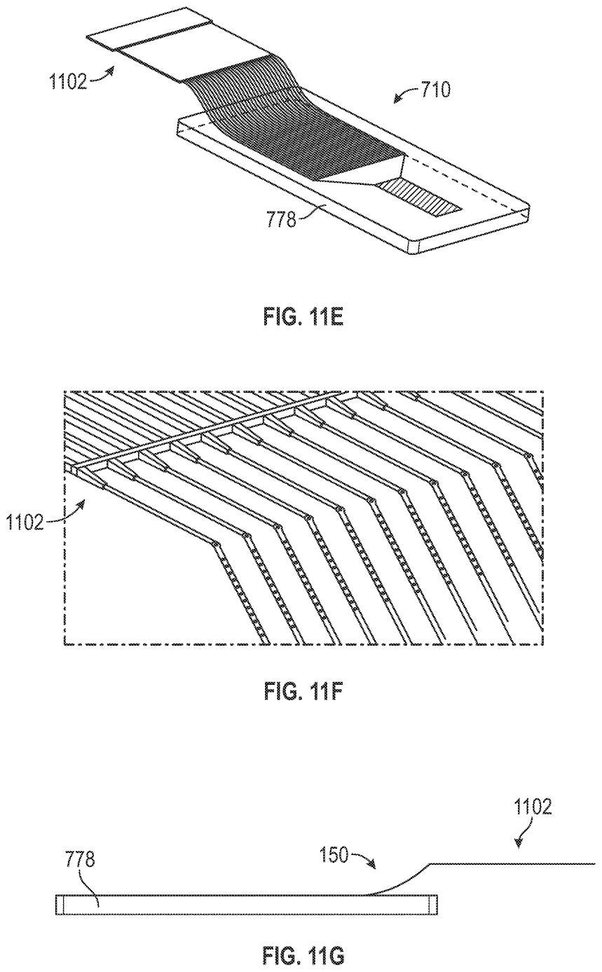

As stated, stage 1 process B can be expanded to three different strategies, outlined below. Note, stage 1 process B strategy 1 is discussed in greater detail with respect to FIGS. 6 and 7A-7I. Stage 1 process B strategy 2 is discussed in greater detail with respect to FIGS. 8 and 9A-9K. Stage 1 process B strategy 3 is discussed in greater detail with respect to FIGS. 10 and 11A-11G.

### Strategy 1B1—Assembly From All-Laser Machined Needles

At stage 1B.1.2, the 32-shank flexible neural electrode probe is released and temporarily fixed on a jig and all flexible neural electrodes are aligned and held by a flat substrate.

At stage 1B.1.3, the 32 shanks are aligned with the 32 micro needles. The tips of microneedles are engaged in the holes at the tip of each flexible neural electrode.

At stage 1B.1.4, the microneedles are controlled to move forward and pick up the flexible neural electrodes from the holder substrate.

At stage 1B.1.5, a dissolvable bio-adhesive is applied to secure the flexible neural electrodes to the microneedles.

In this process, the flexible neural electrode/needles assembly is a stand-alone package that can be conveniently handled and shipped.

### Strategy 2—Assembly From Straight Needles and Bent Tubings

At stage 1B.2.1, the 32 micro needles are pre-mounted on a needle rack and fixture with defined inter-needle spacing (e.g. 500 μm). The needles are laser machined from straight tungsten wires.

At stage 1B.2.2, the 32-thread flexible neural electrode probe is released and temporarily fixed on a jig and all flexible neural electrodes are aligned and held by a flat substrate.

At stage 1B.2.3, the 32 flexible neural electrodes are aligned with the 32 micro needles. The tips of micro needles are engaged in the holes at the tip of each flexible neural electrode. The micro needles are then controlled to move forward and pick up the flexible neural electrodes from the holder substrate. A dissolvable bio-adhesive is applied to secure the flexible neural electrodes to the micro needles.

At stage 1B.2.4, the rear ends of the micro needles are inserted into and bonded with bent tubings.

At stage 1B.2.5, the flexible neural electrode/needles/tubings assembly is now a stand-alone package that can be fixed on a mounting platform and can be conveniently handled and transported.

At stage 1B.2.6, the manipulator arms are aligned with the upper part of the tubings, and bond together with adhesive. The needle rack and mounting platform are then removed.

### Strategy 3—Assembly From Interconnected Needles and Bent Tubings

At stage 1B.3.1, the 32 micro needles are laser machined from sheet metal with precisely defined inter-needle spacing (e.g. 500 μm). The inter-needle spacing matches precisely with the inter-thread spacing of a flexible neural electrode probe.

At stage 1B.3.2, the 32-shank flexible neural electrode probe is released and temporarily fixed on a jig and all flexible neural electrodes are aligned and held by a flat substrate.

At stage 1B.3.3, the 32 shanks are aligned with the 32 micro needles. The tips of microneedles are engaged in the holes at the tip of each flexible neural electrode. The micro needles are then controlled to move forward all together and pick up all the flexible neural electrodes from the holder substrate simultaneously. A dissolvable bio-adhesive is applied to secure the flexible neural electrodes to the micro needles.

At stage 1B.3.4, the rear ends of the micro needles are inserted into and bonded with laser-machined fingers or bent tubings.

At stage 1B.3.5, the flexible neural electrode/needles/tubings assembly is now a stand-alone package that can be fixed on a mounting platform and can be conveniently handled and transported.

At stage 1B.3.6, the manipulator arms are aligned with the upper part of the tubings, and bond together with adhesive. The needle rack and mounting platform are then removed.

In Stage 2, the flexible neural electrodes are implanted using the surgical system.

### Stage 2—Operations Performed During Surgery

At stage 2.1, the surgeon performs a craniotomy and durotomy if needed (skull opening to expose brain tissue).

At stage 2.2, the surgeon aligns the craniotomy site under the micro needles.

At stage 2.3, the laser scanner scans the needles and the brain surface, returning the accurate XYZ coordinates of every needle relative to the brain surface.

At stage 2.4, a camera captures a top-view image of the brain surface and the needles.

At stage 2.5, register the image with the XY coordinates.

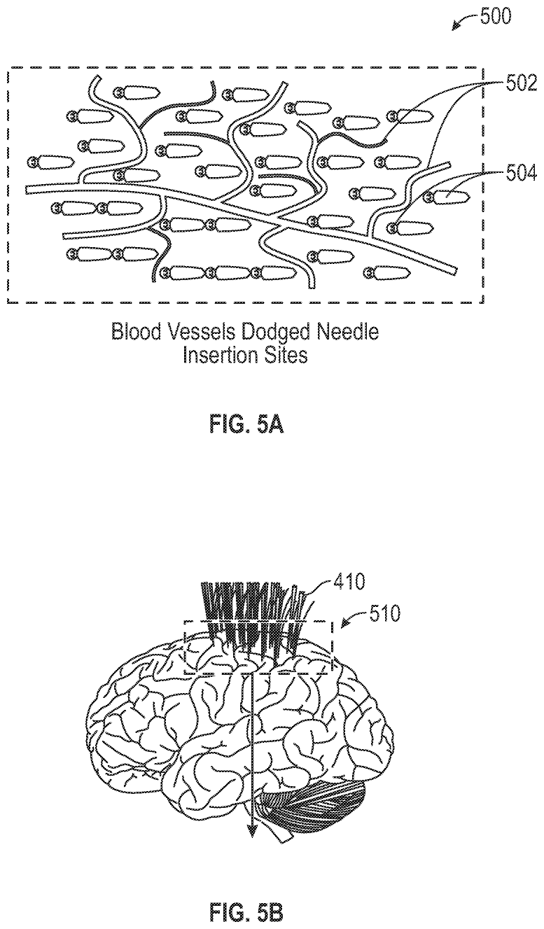

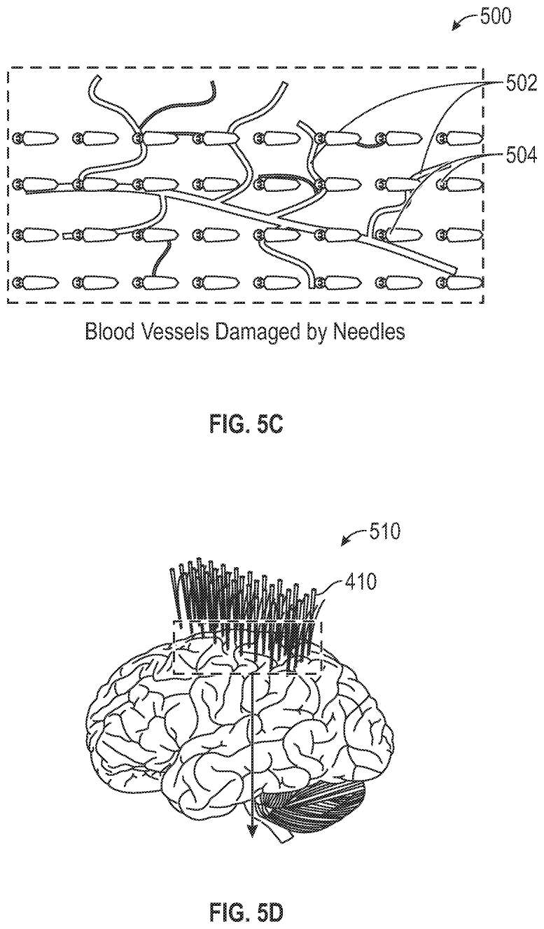

At stage 2.6, the surgeon chooses implantation sites and marks them in the image where to insert (FIGS. 5A and 5B).

At stage 2.7, the surgeon decides the insertion sequence, insertion speed and depth for each thread.

At stage 2.8, start insertion.

At stage 2.9, after all flexible neural electrodes are in place, the Z actuators rapidly retract the micro needles, leaving the flexible neural electrodes inside brain tissue.

In accordance with one or more embodiments, stage 2 can be expanded (i.e., “2+”) as follows.

### Stage 2+—Operations Performed During Surgery

At stage 2+.1, the surgeon performs a craniotomy (and durotomy, if needed) (skull opening to expose brain tissue).

At stage 2+.2, the surgeon aligns the craniotomy site under the micro needles.

At stage 2+.3, the laser scanner scans the needles and the brain surface, returning the accurate XYZ coordinates of every needle relative to the brain surface.

At stage 2+.4, the camera-beamsplitter system captures an image of the brain surface and the needles overlayed (FIGS. 3A and 3B).

At stage 2+.5, register the image with the XY coordinates.

At stage 2+.6, the surgeon chooses implantation sites and marks them in the image where to insert (FIGS. 5A and 5B).

At stage 2+.7, the surgeon decides the insertion sequence, insertion speed and depth for each thread.

At stage 2+.8, start insertion.

At stage 2+.9, after all flexible neural electrodes are in place, the Z actuators rapidly retract the micro needles, leaving the flexible neural electrodes inside brain tissue.

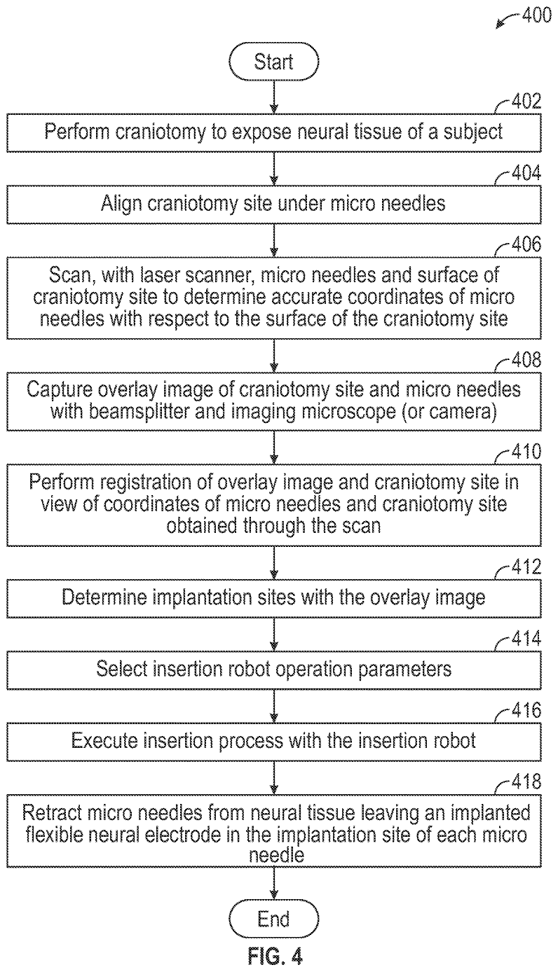

FIG. 4 describes a method of using a surgical system (100) as previously described to implant one or more flexible neural electrodes (150) into neural tissue (198) using an insertion robot (110). Specifically, FIG. 4 provides more details for the expanded stage 2 operations as listed above. FIG. 5A depicts a determination of implantation sites corresponding to a step of the method of FIG. 4. FIG. 5B depicts the insertion of multiple flexible neural electrodes according to the insertion sites of FIG. 5A. In contrast, FIGS. 5C and 5D, for contrast, depict a scenario where the insertion robot is not used to manipulate the micro needles to determined insertion sites, resulting in damaged neural tissue (e.g., pierced blood vessels). The use of the insertion robot (110) may require the threading or the establishing of a releasable connection between the flexible neural electrodes (150) and the micro needles (140). As such, FIGS. 6-11G describe and depict various methods for threading flexible neural electrodes (150) and micro needles (140) and/or fabricating a micro needle and flexible neural electrode assembly (“needle/flexible neural electrode assembly”) for use with the insertion robot (110), as described herein. That is, the various methods depicted and described with respect to FIGS. 6-11G can be used to fabricate a needle/flexible neural electrode assembly. In the examples of FIGS. 6-11G, a nanoelectronic thread (NET) is used as a flexible neural electrode. Thus, in some instances, terms such as needle/NET assembly may be used. However, one with ordinary skill in the art will appreciate that embodiments of the instant disclosure are applicable to many types of flexible neural electrodes, including nanoelectronic threads, such that the use of nanoelectronic threads in these examples is non-limiting.

Turning to the method (400) of FIG. 4, in Step 402 an operator (e.g., surgeon) or operator's assistant (e.g., clinical staff) performs a craniotomy to expose neural tissue of a subject (e.g., expose the brain of a rodent). In Step 404, the operator aligns the craniotomy site (neural tissue) under the micro needles of the insertion robot, where it is assumed that a flexible neural electrode is threaded or releasably connected to each micro needle. An example of his alignment can be seen in FIG. 3A where the micro needles are seen above a calibration sample. However, in this case it is further assumed that a calibration of the micro needles and image acquired using an imaging microscope (or camera) has been previously performed such that the craniotomy site is placed under, or aligned with, the micro needles. In Step 406, the laser scanner is used to scan all the micro needles and surface of the craniotomy site (i.e., neural tissue) to determine accurate coordinates for each micro needle relative to the surface of the craniotomy site. In Step 408, the imaging microscope (or camera) and beamsplitter are used to capture an overlay image of the craniotomy site with the micro needles. An example of an overlay image is provided in FIG. 3B; however, it is noted that in FIG. 3B the overlay image depicts a calibration sample as opposed to a craniotomy site (i.e., neural tissue). In Step 410, a registration of the overlay image of the craniotomy site and the coordinates of the micro needles is performed. That is, coordinates of the micro needles determined using the laser scanner are applied to the image. In Step 412, implantation sites on the neural tissue (i.e., within the craniotomy site) are determined. In some embodiments, implantation sites are determined by selection by an operator via inspection of the image of the craniotomy site. In other embodiments, implantation sites are determined by the controller of the surgical system including the insertion robot through analysis of the image of the craniotomy site, for example, using object detection techniques such as machine learning. In this case, an implantation site determined by the controller can be confirmed and/or monitored during insertion of a flexible neural electrode by the operator. Generally, implantation sites—whether selected by an operator or automatically by the controller—are determined to mitigate damage to the neural tissue, e.g., by avoiding implantation in or near a blood vessel.

FIG. 5A depicts the determination of implantation sites (504) in accordance with one or more embodiments. Specifically, FIG. 5A shows an image of a craniotomy site (500). Multiple bloods vessels (502) can be seen in the image of the craniotomy site (500). In FIG. 5A, 32 implantation sites (504) have been determined where the implantation sites (504) do not intersect or interact with the blood vessels (502). Note that in FIG. 5A, to avoid cluttering the figure and to promote clarity, not all of the blood vessels (502) and implantation sites (504) are labelled. Returning to FIG. 4, in Step 414 the operator chooses various operation parameters of the insertion robot such as: an insertion sequence (where at least two sets of micro needles, each set containing at least one micro needle, are inserted in an ordered sequence) or parallel and simultaneous insertion; insertion speed (can be specified for each micro needle); and insertion depth (can be specified for each micro needle). In Step 416, with the operation parameters chosen, the insertion process is executed by the insertion robot. FIG. 5B depicts craniotomy site 510 and the translation of each micro needle 140 to a location directly above its corresponding implantation site (504) as determined in Step 412. That is, in one or more embodiments, actuators of the insertion robot move the micro needles to form a pattern according to the determined implantations sites (504). Keeping with FIG. 4, in Step 418 after all the micro needles have been inserted in the neural tissue at their corresponding implantation sites (504), the micro needles are retracted rapidly such that the flexible neural electrodes remain implanted in the neural tissue. In review, during insertion, each micro needle (140) may anchor one flexible neural electrode (150) and drive the flexible neural electrode (150) into neural tissue (198). Since the micro needles (140) may move independently, the operator (e.g., surgeon) has the freedom to choose whether to insert them simultaneously or with a certain sequence. After insertion, the micro needles (140) retract from the neural tissue (198) rapidly, leaving the flexible neural electrode (150) implanted in the neural tissue (198).

FIGS. 5C and 5D, in contrast, depict the location of implantation sites assuming an array of 4 by 8 micro needles is used (e.g., without the insertion robot (110)) to implant the flexible neural electrodes. As seen in FIG. 5C, there are various locations where an implantation site (504) intersects a blood vessel (502) causing damage to the neural tissue. FIG. 5D depicts the uniform array of micro needles (140) being uncompliant to the locations of the blood vessels in craniotomy site 510.

As stated, the use of the insertion robot (110) may require the threading or the establishing of a releasable connection between the flexible neural electrodes (150) and the micro needles (140). As previously stated, FIGS. 6-11G depict various methods for fabricating a needle/flexible neural electrode assembly. In particular, these examples use a nanoelectronic thread (NET) as a flexible neural electrode. Thus, in these instances, a needle/flexible neural electrode assembly may be referred to as a “needle/NET assembly,” or variant thereof. That is, while the following examples depict the use of a nanoelectronic thread, this should not impose a limitation on the instant disclosure. In general, the following methods can be readily applied to fabricate a generalized needle/flexible neural electrode assembly. That is, a needle/NET assembly is given as an example needle/flexible neural electrode assembly using a NET as a flexible neural electrode.

FIG. 6 describes a method (600) for fabricating a micro needle and NET assembly (“needle/NET assembly”) for use with the insertion robot (110) (i.e., stage 1, process B, strategy 1), as described herein. In FIG. 6, in Step 602, N micro needles with a specified geometry (e.g., the geometry depicted in FIG. 7A), are provided by laser-machining the micro needles in the specified geometry from a metal sheet, where N is an integer greater than or equal to one. In one or more embodiments, N is set equal to a number of NETs provided on a NET probe. A NET probe (710) is depicted in FIG. 7B. The NET probe (710) consists of one electronic backend connector (778) and a specified number (e.g., N) of NETs (150). The electronic backend connector (778) contains bonding pads that transmit neural electrophysiology signals to a data collection device (e.g. a computer). The NETs (150) are designed to be implanted in the neural tissue, containing microelectrodes that record neural electrophysiology signals and apply electrical stimulation. The NETs are made of polyimide, of thickness ranging from 1 μm to 2 μm. In some embodiments, N is set equal to 32. That is, the NET probe (710) contains 32 NETs with a defined inter-thread spacing.

Returning to FIG. 7A, FIG. 7A depicts a specified geometry for the laser-machined micro needles (702). As seen, the laser-machined micro needles (702) have two bends where each bend is substantially 90 degrees. As described below, the laser-machined micro needles (702) are configured for placement in a needle fixture (704) and a first needle rack (706). Specifically, the first needle rack (706) holds the distal ends of the laser-machined micro needles (702) in a linear array or arrangement with uniform spacing between the needles (e.g., 500 μm inter-needle spacing). The needle fixture (704) holds the proximal ends of the laser-machined micro needles (702) in a pre-defined pattern (e.g., a pattern that the distal ends of the manipulator arms of the insertion robot can assume) for convenient connection of the laser-machined microneedles to the manipulator arms at a later time.

Keeping with FIG. 6, in Step 604 the laser-machined micro needles (702) are mounted in a needle fixture (704) and a first needle rack (706). FIGS. 7C, 7D, and 7E depict a front view (701), a side view (703), and a top view (705) of laser-machined micro needles (702) mounted in a needle fixture (704) and first needle rack (706), respectively. In some embodiments, the needle fixture (704) and first needle rack (706) are two separate elements that can be connected as seen in FIGS. 7C-7E. In other embodiments, the needle fixture (704) and first needle rack (706) are a single component.

Returning to FIG. 6, in Step 606 a NET probe (710) containing N NETs is temporarily affixed to a jig (708) where all the NETs of the NET probe (710) are aligned and held flat by a substrate (712). The jig (708), NET probe (710), and substrate (712) are depicted in FIGS. 7F and 7G, where these figures provide alternative views, in accordance with one or more embodiments. In Step 608, the jig (708) and first needle rack (706) are aligned such that holes provided in the distal ends of the NETs are engaged in the tips of the laser-machined micro needles (702). This is facilitated by the inter-needle spacing of the laser-machined micro needles (702), when mounted in the first needle rack (706), being the same as the spacing between NETs on the NET probe (710), FIG. 7H depicts a hole (714) provided in the distal end of a NET (150) and a laser-machined micro needle (702) engaging in the hole (714).

In Step 610, the first needle rack (706) (and, in some instances, also the needle fixture (704)) and jig (708) are fixed relative to each other. With the needle rack (706) and jig (708) fixed relative to each other, the laser-machined micro needles (702) are slid along the needle rack (706) to thread with the NETs such that the engaged laser-machined micro needles (702) pick up the NETs (150) of the NET probe (710) from the substrate (712). After threading, the needle rack (706) and/or needle fixture (704) can be tightened to prevent further movement of the laser-machined micro needles (702) relative to the needle rack (706) fixing the position and orientation of the now-threaded laser-machined micro needles. Finally, in Step 612 a dissolvable bio-adhesive is applied to the NETs and distal ends of the laser-machined micro needles to secure the NETs to the laser-machined micro needles (702). The product of the method (600) of FIG. 6 is a needle/NET assembly (720). FIG. 71 depicts the needle/NET assembly (720) in accordance with one or more embodiments. As seen, the needle/NET assembly (720) consists of the first needle rack (706), needle fixture (704), N laser-machined micro needles (702) each with an attached NET (150), FIG. 7I further depicts the non-distal ends of the NETs (150) connected to a NET backend connector (778) for interfacing with an external system such as a computer. The needle/NET assembly (720) is a stand-alone package that can be conveniently handled and shipped. That is, the needle/NET assembly (720) is ready for use with an insertion robot (110) as previously described.

FIG. 8 describes a method (800) for fabricating a micro needle, NET and bent tubing assembly (“needle/NET/tubing assembly” or “NET cartridge”) for use with the insertion robot (110) (i.e., stage 1, process B, strategy 2), as described herein. The system design for method (800) is shown, at least in part, in FIGS. 9A-9I. Thus, to facilitate the description, an image of the needle/NET/tubing assembly (910) is depicted in FIG. 9A. The needle/NET/tubing assembly (or NET cartridge) is designed as a standalone module that can be conveniently transferred among different locations. The flexible threads of NET (150) are pre-threaded on the tungsten wire micro needles (904), thus eliminating the necessity of performing the delicate threading process during surgical operation.

As shown in FIGS. 9A-9I, in accordance with one or more embodiments, the needle/NET/tubing assembly consists of five major components. Component 1 includes a 32-thread NET probe (710): an electrode array with one electronic backend connector (778) and 32 NETs (150). Each thread will ultimately be implanted in the neural tissue. Component 2 includes tungsten wire micro needles (904), where each micro needle anchors one thread mechanically, and ultimately will drive and insert the thread into brain. After insertion, the micro needles will be retracted from the neural tissue. Component 3 includes a needle mounting cap (902) that is a solid structure with micro grooves (950) that restrict (together with the needle mounting base (903)) the movement of the micro needles in one dimension. Component 4 includes a needle mounting base (903) that is a solid structure with micro grooves (950) that restrict (together with the needle mounting cap (902)) the movement of the micro needles in one dimension. Component 5 includes bent tubings (271) that are 32 tubes that connect the micro needles and the manipulator arms of an insertion robot.

FIG. 9B further depicts the needle/NET/tubing assembly (910) in a mounting platform (908). FIG. 9C depicts an enlarged view of the needle/NET/tubing assembly (910). FIG. 9D depicts a NET probe (710) with its NETs (150) attached to tungsten wire micro needles (904) supported by the needle mounting cap (902) and needle mounting base (903). FIG. 9E depicts a front view of the mounting base (903) and FIG. 9F depicts a back view of the mounting cap (902). As seen in both FIGS. 9E and 9F, both the mounting base (903) and the mounting cap (902) have micro grooves (950). In one or more embodiments, the micro grooves (950) have a width of 0.1 mm and a 0.5 mm pitch. The micro grooves (950) are depicted in FIG. 9G. When the mounting cap (902) and mounting base (903) are joined together, the micro grooves (950) of the cap and base will form 32 enclosed tunnels. Each micro needle will be clamped within one tunnel-the needle can only move back and forth along the tunnel's longitudinal direction as depicted in FIG. 9H. FIG. 9I depicts a side of the micro needles within the tunnels formed by the joining of the mounting cap (902) and the mounting base (903).

Returning to FIG. 8, in Step 802, M straight tungsten wire micro needles are provided by laser-machining, where M is an integer greater than or equal to one. Further, in Step 802, a second needle rack with a defined inter-needle spacing (e.g., 500 μm) is also provided. The second needle rack may be identical to the first needle rack as described with respect to FIG. 6. In one or more embodiments, M is set equal to a number of NETs provided on a NET probe. In some embodiments, M is set equal to 32.

In Step 804 the tungsten wire micro needles are mounted in the mounting cap (902) and the mounting base (903) which may be considered a second needle rack. In some embodiments, the first needle rack, as previously discussed, is identical to the second needle rack. That is, the first needle rack can be composed of a mounting cap and a mounting base. In Step 806 a NET probe (710) containing M NETs is temporarily affixed to a jig (708) where all the NETs of the NET probe (710) are aligned and held flat by a substrate (712). The jig (708), NET probe (710), and substrate (712) are depicted in FIGS. 7F and 7G, in accordance with one or more embodiments. In Step 808, the jig (708) and second needle rack (i.e., combination of mounting cap and mounting base) are aligned such that holes provided in the distal ends of the NETs are engaged in the tips of the tungsten wire micro needles. This is facilitated by the inter-needle spacing of the tungsten wire micro needles, when mounted in the second needle rack, being the same as the spacing between NETs on the NET probe (710). In some embodiments, the second needle rack is fixed relative to the jig (708).

In Step 810, single micro needles are moved (or slid) in the second needle rack such that the engaged tungsten wire micro needles pick up the NETs (150) of the NET probe (710) from the substrate (712). FIGS. 9J and 9K depict the process of engaging a tungsten wire micro needle (904) with the a NET (150) using the mounting cap (902), mounting base (903), and jig (708). Engagement of the micro needles to the NETs is also known as a threading procedure or threading. Threading is designed to be performed on a wet bench before surgical operation. The purpose of the threading procedure is to fix each NET and each micro needle together, forming a convenient ready-to-use assembly. In some embodiments, once each micro needle has been threaded with a NET, the second needle fixture is tightened to prevent further movement of the micro needles in the second needle rack. Returning to FIG. 8, in Step 812, a dissolvable bio-adhesive is applied to the NETs and distal ends of the tungsten micro needles to secure the NETs to the tungsten micro needles. Finally, in Step 814 the proximal ends of the tungsten wire micro needles are bonded with bent tubings. The product of the method (800) of FIG. 8 is a needle/NET/tubing assembly (910). FIG. 9A depicts the needle/NET/tubing assembly (910) in accordance with one or more embodiments. As seen, the needle/NET/tubing assembly (910) consists of the mounting cap (902), mounting base (903), M laser-machined tungsten wire micro needles (904) each with an attached NET (150), and bonded bent tubing (271) to the proximal end of each tungsten wire micro needle (904). The needle/NET/tubing assembly (910) is a stand-alone package that can be conveniently handled and transported. Further, the needle/NET/tubing assembly (910) can be fixed on mounting platform for connection of the bent tubings (271) to an insertion robot (110). In one or more embodiments, the needle/NET/tubing assembly (910) is mounted on a mounting platform (908) as depicted in FIG. 9B. While on the mounting platform (908) and proximate to an insertion robot (110), manipulator arms of the insertion robot (110) can be aligned with the upper or proximal ends of the bent tubings (271) and bent tubings (271) and manipulator arms can be bond together with adhesive. Once the bent tubings (271) and manipulator arms are adhered, the mounting cap, mounting base, and mounting platform (908) can be removed readying the insertion robot (110) for operation.

FIG. 10 describes a method (1000) for fabricating an interconnected micro needle and NET assembly (“interconnected needle/NET assembly”) for use with the insertion robot (110) (i.e., stage 1, process B, strategy 3), as described herein. In FIG. 10, in Step 1002, K micro needles are provided by laser-machining sheet metal, where K is an integer greater than or equal to one. The K micro needles are interconnected, as shown in FIG. 11A, and have a defined inter-needle spacing, i.e., interconnected micro needles (1102). The interconnects of the interconnected micro needles (1102) provide rigidity and ensure the defined inter-needle spacing. In one or more embodiments, K is set equal to a number of NETs provided on a NET probe (710). In some embodiments, K is set equal to 32. FIGS. 11B and 11C show progressively zoomed in or enlarged views of the distal ends of the interconnected micro needles (1102). In one or more embodiments, the interconnected micro needles (1102) are laser-machined to have post (1104) at their distal ends. The post (1104) is designed to enter a hole provided at the distal end of a NET (150).

Returning to FIG. 10, in Step 1004 a NET probe (710) containing K NETs is provided with its electronic backend connector (778) where all the NETs of the NET probe (710) are aligned. The electronic backend connector (778), NET probe (710), and interconnected micro needles (1102) are depicted in FIG. 7D, in accordance with one or more embodiments. In Step 1006, the NET probe (710) and interconnected micro needles (1102) are aligned such the posts (1104) of the interconnected micro needles (1102) penetrate the holes provided in the distal ends of the NETs. This is facilitated by the inter-needle spacing of the interconnected micro needles (1102) being the same as the spacing between NETs on the NET probe (710). In some embodiments, the NET probe (710) is affixed to a jig (708) as previously described. In these instances, the NETs of the NET probe (710) are aligned with the interconnected micro needles (1102) using the jig (708).

In Step 1008, the interconnected micro needles (1102) are moved relative to the NET probe (710) pick up the NETs (e.g., from a substrate or from the electronic backend connector (778)). In step 1010, a dissolvable bio-adhesive is applied to the NETs and distal ends of the interconnected micro needles (1102) to hold the NETs to the interconnected micro needles (1102). Finally, in Step 1012 the proximal ends of the interconnected micro needles (1102) are inserted into and bonded with laser-machined fingers or bent tubings for eventual connection with the manipulator arms of an insertion robot (110). The product of the method (1000) of FIG. 10 is an interconnected needle/NET assembly (1110). FIG. 11D depicts a top view of the interconnected needle/NET assembly (1110) along with the jig (708) supporting the NET probe (710) including the NETs (150) where the NETs (150) are connected to the interconnected micro needles (1102), in accordance with one or more embodiments. The interconnected needle/NET assembly (1110) is a stand-alone package that can be conveniently handled and transported.

FIG. 11E depicts the interconnected needle/NET assembly in accordance with one or more embodiments. FIG. 11F depicts an enlarged view of the engagement of the interconnected micro needles (1102) and the NETs. FIG. 11G depicts a side view of the interconnected needles/NET assembly as the interconnected needles are moved to pick up the NETs.

FIG. 12 is a block diagram depicting the interaction and electrical communication of various components of the surgical system (100), in accordance with one or more embodiments.

FIG. 12 depicts a computer (1202) including a processing unit (1212). The processing unit (1212) executes stored code or software to communicate with, control, tune and read from the different hardware components making up the robotic strategy for insertion of flexible neural electrodes, as previously described. In one or more embodiments, the code or software is written in Matlab and Python, but the General-Purpose Language (GPL) for use with the methods and systems described herein is not limited to these languages.

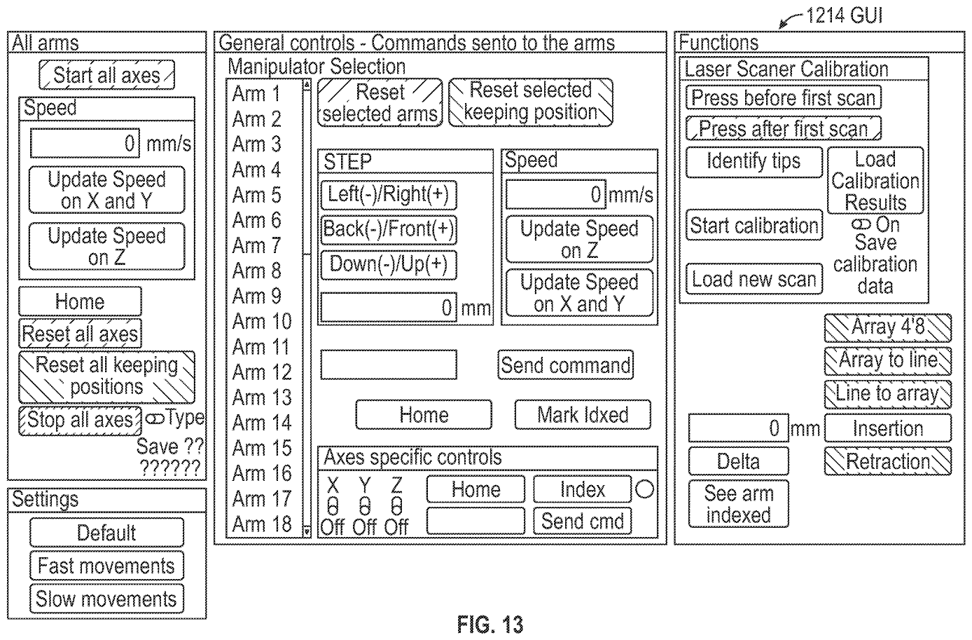

The graphical user interface (GUI) 1214 is the front-end interface that allows an operator of the surgical system (100) to perform insertions of flexible neural electrodes. An example of the GUI (1214) is depicted in FIG. 13. The GUI (1214) incorporates all necessary functionalities to conduct insertion of flexible neural electrodes from threading to insertion to retraction of the micro needles. The GUI (1214) need not be as depicted in FIG. 13 and may include different buttons and functionalities. For example, in one or more embodiments, the GUI (1214) also displays a graphical representation of the coordinates (e.g., position on the x, y, and z axes) of each manipulator arm (e.g., the distal end) and/or supported micro needle. The GUI (1214) can be used to implement the following functions with reference to components depicted in FIG. 12.

Function 1 includes communication initialization with the controllers (1204) of the piezo motors (1206). Note that these controllers are unique to the motors and should not be conflated with the controller (120) of FIG. 1A. Each controller (1204) includes a microcontroller that interfaces with a domain specific language (DSL) of the motor (1208) (i.e., a DSL specific to the motors used in the insertion robot). The motor DSL enables control and tuning of the motors (1206) by the processing unit (1212). It serves as an interface between Python and the commands necessary to interact with the motors (1206). The microcontroller receives data from the motor DSL (1208) and then distributes all the commands to the motors (1206). The controller (1204) is connected to the computer (1202) using USB. The controller (1204) is connected to up to 12 individual motors (1206) using a 15 pin cable. In one or more embodiments, there are 8 controllers (1204) connected to the computer (1202) (controlling a total of 96 motors (1206)), but more could be added if needed. The controller (1204) sends data to the motor DSL (1208) continuously when the communication is started. The 96 motors (1206) are assembled in bundles of three for movements in horizontal, longitudinal, and vertical directions of 32 manipulator arms.

Function 2 includes initialization of parameters of each individual motor (1206) through the motor Domain Specific Language (DSL) (1208) enabling communication between the processing unit (1212) and the controllers (1204).

Function 3 includes overwriting and reading from motor parameters files (1210). Motor parameter files (1210) may include one or more motor parameter files. Parameters from motor parameters files (1210) are sent to the motors (1206) at communication initialization. Parameters in the motor parameters files (1210) can be modified before starting the processing unit (1212) by modifying text files. Parameters in the motor parameters files (1210) can also be modified by the processing unit (1212) during surgical procedures. This can be initiated by the operator or automatically done by the processing unit (1212) during common procedures (for example during retraction). The parameters sometimes need to be modified during the course of an insertion procedure as some steps require different response profiles from the motors (1206).

Function 4 includes manual motor controls. Manual motor controls can include:
a. Motor movements to move each of the 32 manipulator arms in 3 axes (horizontal, longitudinal, vertical or x, y, and z).
b. Speed control of each motor.
c. Homing function to return to position of origin (position the motor had at start up).
d. Indexing to find the position of each individual stage in real space (by default each motor is initialized at software position 0 even when it is not at the center of the travel range).
e. Reset motors when they encounter an error.
f. Send specific commands from a manufacturer provided list through a text input prompt.

All the controls mentioned above can be applied to a single motor, a single arm (the 3 motors attached to a single arm), a selection of arms (from one to all motors), a selection of motors for several arms (for instance to move a selection of arms along one direction only), or all the motors (hence all the arms).

Function 5 includes motors data input. The back-end program consistently reads input from the motors over the COM port. Data is converted from the motor DSL (1208) into real word unit (mm or μm). This data includes a variety of information (current position of the motor, desired position, error encountered, etc.). The processing unit (1212) collects the position of the motors (1206) to display a real-time visual three-dimensional representation of all micro needles to the GUI (1214).

Function 6 includes micro needle (or surgical wire) registration. Using three-dimensional point cloud data obtained by the laser scanner (160), micro needle or wire positions relative to each other and relative to a reference (e.g., neural tissue surface) can be determined. These relative positions are then fed into the processing unit (1212) that updates the real space positions of the micro needles or wires on the three-dimensional representation discussed above. Real space here denotes a difference from software space, that only includes the positions given by the motors (1206) themselves, indicating the current positions relative to where motors (1206) were located when turned on and not necessarily where they are positioned within their full travel range. The processing unit (1212) combines laser scans and real-time input from the motors (1206) to provide a live three-dimensional representation of the micro needle or wire arrangement and neural tissue surface to precisely plan flexible neural electrodes insertion. In general, laser scanning only needs to be done before the insertion of flexible neural electrodes. Both the laser scanner (160) and the laser linear stage (1220) each have a DSL (in Python) making it possible for the processing unit (1212) to send commands and retrieve laser scans data. Specifically, the laser linear stage (1220) is controlled by Zaber Software (1216) but also has a DSL that can be interfaced using Python so the laser linear stage (1220) can be controlled by the processing unit (1212). The laser scanner (160) is commanded by the manufacturer's software (1218) and can also be controlled using a DSL by the processing unit (1212). For example, the manufacturer's software (1218) may be the software of LJ-X8000A laser profiler commercially available from Keyence. It communicates with the main computer (1202) using an ethernet cable. Thus, it is possible to have a fully automated micro needle or wire identification process on the GUI (1214) as long as the laser scanner (160) is properly placed in front of the micro needles or wires, or the process can be performed outside the processing unit (1212) using the two softwares independently.

Function 7 includes automatic movements of the micro needles or surgical wires. Once the real space position of the wires is obtained, the processing unit (1212) automatically calculates the movements of each motor (1206) necessary to bring the micro needles or wires in a predefined desired arrangement (few examples include: a line for threading, a 4 by 8 array, a 2 by 16 array, etc.).

Function 8 includes visual feedback. The beamsplitter camera system (1226) (e.g., as depicted in FIG. 3A), as well as an optional real time broadcast of the surgical site can be displayed on the GUI, or as standalone programs outside the processing unit (1212). Using beamsplitter images (e.g., FIG. 3B) imported to the processing unit (1212), the operator is able to align the micro needles or wires with brain vasculature to target specific insertion sites while avoiding blood vessels. An artificial neural network (e.g., 3D Convolutional Neural Network or U-Net) can be used to automatically segment brain images and isolate blood vessels from tissue to prevent accidental puncture of blood vessels, detrimental to neural recordings and resulting in brain damage. The operator can select insertion sites from the beamsplitter images or perform automatic insertion at random but sparse locations using blood vessels avoidance features, where locations of the blood vessels are determined using the segmented images. As discussed above, the beamsplitter is removed during the insertion as it sits between the neural tissue and the micro needles or wires. This is when another camera (e.g., imaging microscope) can be used to display a live imaging of the insertion of micro needles in neural tissue if necessary.

Function 9 includes insertion protocols. To insert the flexible neural electrodes in neural tissues, the operator can simply click a button on the front-end of the GUI (1214) that will lower the micro needles and flexible neural electrodes in the neural tissue. The back end will change parameters in the motor parameters files (1210) to adapt to the requirements of a successful insertion (meaning minimizing tissue scaring) prior to lowering the micro needles into the neural tissue. Insertion depth can be tuned altogether or individually for each arm. Insertion sequence can also be tuned by the operator (simultaneous insertion or sequential insertion).

Function 10 includes retraction protocols. Once the flexible neural electrodes have been successfully inserted into the neural tissue, parameters in the motor parameters files (1210) will be automatically changed to allow fast and steady retraction of the micro needles. High acceleration is important to ensure that the implantable flexible neural electrodes do not come out with the micro needles or wires when retracting. Parameters are also tuned to reduce vibration that could induce tissue damage.

Function 11 includes communication closure.

Thus, for example, embodiments disclosed herein relate to system for implanting neural electrodes (e.g., NETs), where the system includes: an insertion robot including a plurality of independently controllable end effectors, each configured to insert a neural electrode into neural tissue, with at least some of the insertions being performed in parallel. The insertion robot further includes a plurality of robotic arms (e.g., manipulator arms) where each of the robotic arms supports one of the end effectors and each end effector can include a micro needle configured to insert the neural electrode into the neural tissue. The micro needles are independently movable in three translational degrees of freedom, driven by actuators. In one or more embodiments, each of the actuators is a piezoelectric motor. In one or more embodiments, the cardinality of the plurality of independent controllable end effectors is 32. Further, in one or more embodiments, the neural electrode is a flexible neural electrode. The system can further include a laser scanner configured to operate as a position sensor configured to determine, for each neural electrode, a position relative to a surface of the neural tissue. The system can further include an imaging microscope configured to provide live visual feedback of the insertion to an operator (e.g., surgeon).

Embodiments of the disclosure may have various applications such as brain-computer interfaces, diagnosis and treatment of neurological disorders, fundamental neuroscience research, etc. Embodiments of the disclosure allow implantation of flexible neural electrodes that would otherwise be time-consuming and, when performed by humans, would have poor reliability due the scale at which the surgeries are performed (i.e., small workspace, small working area or working volume) and the fragility of flexible electrodes. A large number of flexible neural electrodes may be simultaneously implanted at a higher density and a wider variety of angles than previously possible. The system as described can thread flexible neural electrodes, which is primordial for chronic implantations. In addition, the surgical system can implant at closer probe spacing or higher density than previously possible. Independent positioning of each of the flexible neural electrodes enables a superior targeting of desired locations while avoiding undesirable locations such as blood vessels (i.e., determined implantation sites).

Embodiments of the disclosure reduce the need for human input and accelerate the implantation procedures, thereby enabling repeatable and large-scale surgical implantation of flexible neural electrodes. Embodiments of the disclosure may be used for acute implantations or chronic implantations. Multiple, or even many flexible neural electrodes may be inserted (even simultaneously) in neural tissue, which enables recording from larger populations of neurons. With simultaneous implantation of many flexible neural electrodes, the procedures time may be reduced, while targeting multiple brain regions, and substantially reduce surgical injury.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

1. A system for implanting flexible neural electrodes, the system comprising:
an insertion robot comprising:
a base,
a plurality of manipulator arms each configured to support a micro needle,
and a plurality of actuators supported by the base,
wherein each manipulator arm is translatable in three orthogonal directions by a corresponding actuator of the plurality of actuators,
wherein the plurality of manipulator arms is configured to reduce an area spanned by its distal ends relative to an area spanned by the plurality of actuators; and
a controller comprising one or more processors, the controller in electrical communication with the insertion robot and configured to control each manipulator arm in the plurality of manipulator arms to implant, with the micro needle, a flexible neural electrode into neural tissue, with at least some insertions being performed in parallel.

2. The system of claim 1, wherein each actuator in the plurality of actuators comprises three motors arranged in a series, each motor translatable in one of the three orthogonal directions.

3. The system of claim 1, wherein each manipulator arm in the plurality of manipulator arms is independently controllable by the controller, wherein the controller is configured to independently control each manipulator arm in the plurality of manipulator arms.

4. The system of claim 1, wherein a cardinality of the plurality of manipulator arms is 32.

5. The system of claim 1, further comprising:
a laser scanner configured to operate as a position sensor configured to determine, for each manipulator arm, a position of the micro needle supported by the manipulator arm relative to a surface of the neural tissue.

6. The system of claim 1, further comprising:
an imaging microscope configured to provide live visual feedback of the insertions to an operator.

7. The system of claim 1, further comprising a beamsplitter configured to be positioned temporarily between micro needles supported by the plurality of manipulator arms and the neural tissue, wherein the beamsplitter is arranged to produce an overlay image of the neural tissue and the micro needles at an imaging microscope or camera.

8. The system of claim 7, further comprising a light source configured to illuminate the micro needles for distinction in the overlay image.

9. The system of claim 8, wherein the controller is further configured to determine an implantation site on the neural tissue for a micro needle supported by a manipulator arm of the plurality of manipulator arms.

10. The system of claim 9, wherein:
the system further comprises a laser scanner configured to operate as a position sensor configured to determine, for each manipulator arm, a position of a micro needle supported by the manipulator arm relative to a surface of the neural tissue; and
the controller is further configured to register the overlay image based on the position of the micro needle.

11. The system of claim 9, wherein the controller is further configured to determine a planar distance between the implantation site and the micro needle.

12. The system of claim 11, wherein the controller is further configured to position the micro needle to its determined implantation site based on the planar distance.

13. A method for implanting flexible neural electrodes, the method comprising:
scanning, with a laser scanner, a craniotomy site comprising exposed neural tissue and proximate micro needles, forming a laser scan;
determining, based on the laser scan, coordinates of the micro needles with respect to a surface of the craniotomy site;
capturing an overlay image of the craniotomy site and the micro needles using a beamsplitter and an imaging microscope or camera;
performing a registration of the overlay image and the craniotomy site based on the coordinates of the micro needles;
determining implantation sites with the overlay image;
executing an insertion process of the micro needles, each releasably connected to a flexible neural electrode, into the neural tissue according to an insertion sequence; and
retracting the micro needles from the neural tissue leaving, for each micro needle, an implanted flexible neural electrode in the neural tissue at a corresponding implantation site of the micro needle.

14. The method of claim 13, further comprising threading the flexible neural electrodes to the micro needles.

15.-19. (canceled)

20. A method for fabricating a micro needle and flexible neural electrode assembly, the method comprising:
temporarily disposing a flexible neural electrode probe comprising flexible neural electrodes on a jig with the flexible neural electrodes aligned and held by a substrate;
aligning micro needles, interconnected or pre-mounted on a needle rack, with distal ends of the flexible neural electrodes of the flexible neural electrode probe, wherein the micro needles whether pre-mounted or interconnected have a defined inter-needle spacing;
engaging tips of the micro needles in holes at the distal ends of the flexible neural electrodes; and
picking up the flexible neural electrodes from the substrate.

21. The method of claim 20, wherein the micro needles are pre-mounted on the needle rack and the method further comprises:
applying a dissolvable bio-adhesive to secure the flexible neural electrodes to the micro needles.

22. The method of claim 20, wherein aligning the micro needles and the distal ends of the flexible neural electrodes comprises aligning the jig and the needle rack or the interconnected micro needles.

23. The method of claim 20, further comprising:
mounting the micro needles on the needle rack, wherein mounting the micro needles on the needle rack comprises:
providing, by laser-machining a metal sheet, the micro needles with a specified geometry; and
mounting the micro needles in a needle fixture and a needle rack with the defined inter-needle spacing.

24. (canceled)

25. The method of claim 20, further comprising:
mounting the micro needles on the needle rack, wherein the needle rack comprises a mounting base and mounting cap each having a micro groove for each micro needle and mounting the micro needles on the needle rack comprises:
disposing the micro needles in tunnels formed by the micro grooves of the mounting cap and the mounting base through attachment of the mounting cap to the mounting base,
wherein the micro grooves have the defined inter-needle spacing.

26.-32. (canceled)

33. The method of claim 20, further comprising disposing the assembly below manipulator arms of an insertion robot and bonding or attaching proximal ends of the micro needles to the manipulator arms.
